(19) United States
(12) Patent Application Publication
(10) Pub. No.: US 2013/0225427 A1
(43) Pub. Date: Aug. 29, 2013

(54) METHOD FOR PREDICTION OF RESPONSE TO IMMUNE MEDIATED DISEASE THERAPEUTICS

(71) Applicant: Sanford-Burnham Medical Research Institute, (US)

(72) Inventor: Salvatore Albani, Encinitas, CA (US)

(73) Assignee: SANFORD-BURNHAM MEDICAL RESEARCH INSTITUTE, La Jolla, CA (US)

(21) Appl. No.: 13/670,460

(22) Filed: Nov. 7, 2012

Related U.S. Application Data
(63) Continuation-in-part of application No. 13/289,788, filed on Nov. 4, 2011, now abandoned.
Application No. 13/289,788 is a continuation-in-part of application No. 13/229,286, filed on Sep. 8, 2011.
(60) Provisional application No. 61/380,983, filed on Sep. 8, 2010.

(51) Int. Cl. C12Q 1/68; G01N 33/68

(52) U.S. Cl. CPC C12Q 1/6876; G01N 33/68
USPC 506/9; 435/7.1; 435/6.12; 506/16; 506/18

(57) ABSTRACT

The present invention is based in part on the identification of a signature marker profile of immune variables to diagnose an immune mediated disease or for prediction of response to an immune mediated disease therapeutic agent. Additionally, the present invention provides methods for the prediction of response to an immune mediated disease therapeutic agent.

### CROSS REFERENCE TO RELATED APPLICATION(S)

This application claims the benefit of priority under 35 U.S.C. §119(e) of U.S. Ser. No. 61/380,983, filed Sep. 8, 2010, U.S. Ser. No. 13/229,286 filed Sep. 9, 2011, and U.S. Ser. No. 13/289,788, the entire content of which is incorporated herein by reference.

### GRANT INFORMATION

This invention was made in part with government support under Grant # RO1AR056273 awarded by NIAMS. The government has certain rights in the invention.

### BACKGROUND OF THE INVENTION

1. Field of the Invention

The present invention relates generally to the field of immune mediated processes and more specifically to the identification of a signature marker profile of immune variables to diagnose a condition or predict response to a therapeutic agent.

2. Background Information

The immune system is a complex matrix of processes which protects the body from various pathogens. The immune system must recognize a wide variety of pathogens, such as viruses and bacteria, and distinguish those from the body's own cells and tissues. When these processes are disrupted immune mediated diseases may result. Immune mediated diseases are conditions which result from abnormal activity of the body's immune system. The immune system may over react or start attacking the body.

Immune mediated diseases can be divided into several categories including immunodeficiency diseases, autoimmune diseases and hypersensitivity diseases. Immunodeficiency diseases occur when part of the immune system is not functioning properly. Autoimmune diseases are the result of the immune system attacking the body instead of pathogens. Hypersensitivity diseases occur when the immune system over reacts and results in damage to the body.

An example of an autoimmune disease is rheumatoid arthritis (RA). RA is a chronic autoimmune disease that leads to inflammation of the joints and surrounding tissues. The disease is characterized by joint inflammation and pain and usually affects joints in a symmetrical fashion.

The synovial joints are the area principally attacked, producing an inflammatory response of the synovium, hyperplasia of the synovial cells and excess synovial fluid. The cause of RA is unknown and the disease cannot be cured.

Molecular immunology has provided tools for improved knowledge of the mechanisms contributing to the pathogenesis of immune mediated diseases. The chronic inflammatory nature of many of these diseases is now better understood and many of the mediators and pathways that amplify inflammatory processes and lead to tissue damage have been identified. This progress has translated into clinical practice, for example in RA with the introduction of biologic agents that effectively interfere with the inflammatory cascade by blocking one of its key components. Direct biological interference with cytokines, such as TNFa, and accessory molecules on the surface of immune cells, such as synthetic molecules with CTLA-4 or CD3 on T cells or CD20 on B cells, is increasingly replacing generalized pharmacological immune suppression via induction of immune suppression or immune tolerance.

The clinical effectiveness of immune mediated disease therapeutics varies widely depending on the specific condition and the individual subject receiving the therapeutic agent. Altogether, the level of clinical effectiveness, which specifically for RA leaves approximately half of the patients with an insufficient response, and the fact that there are no objective data available to justify the choice of one biologic versus another, underscore the need to improve the knowledge base for current and future therapeutic decisions. Such knowledge should be grounded on a mechanistic-based approach to identify the effects of these drugs on the immune system and their actual mechanism of action in relationship to clinical responsiveness. Awareness is converging from disparate standpoints regarding the fact that biological therapeutic agents—which were approved based on the assumption that they are specific for the immune pathway with which they were designed to interfere—in reality affect a multiplicity of intersecting mechanisms. There is therefore a need to define mechanisms of action of biologic agents with an approach that takes into account effects on organisms and systems rather then on individual pathways. This is also true of many other types of an immune mediated disease therapeutic agents as well, where a patient is subjected to a several different therapeutic agents before identifying one that elicits a positive response. It is precisely here that a fundamental knowledge gap can be identified.

The overarching strategy of the present invention, is that while the clinical features of patients taking various immune mediated disease therapeutic agents will not be significantly different, mechanistic differences between treatments will be distinguishable, thus defining predictors of efficacy based on immune function. For example, effective therapies may induce low disease activity (LDA) in RA by affecting multiple interdependent immune pathways, including, T-cell regulation, lineage commitment (TH-1/17, tolerant/anergic) for effector T cells, and dendritic cell function. The present invention utilizes multiple interacting pathways, rather than a single mechanism, to determine susceptibility to treatment on the basis of immunological regulation. Consequently, it is possible to identify immune functions that cluster with a positive response to an immune mediated disease therapeutic. Such signature marker profiles could contribute to defining the mechanism of action of each treatment and provide a knowledge based, logical framework for clinical decisions, including the choice of a given an immune mediated disease therapeutic for treatment of clinically indistinguishable patients. The tangible translational outcomes from this reverse translational itinerary (bedside to bench) relate not only to a better knowledge of mechanism of action of widely used an immune mediated disease therapeutic agents, but also to the potential use of this newly acquired knowledge to support clinical therapeutic decisions.

### SUMMARY OF THE INVENTION

The present invention is based in part on the identification of a signature marker profile of immune variables to diagnose an immune mediated disease or for the prediction of a response to an immune mediated disease therapeutic agent. Additionally, the present invention provides methods for the prediction of response to such a therapeutics.

Accordingly, in one embodiment, the present invention provides a method to diagnose an immune mediated disease or to predict a response to an immune mediated disease therapeutic agent. The method includes isolating proliferating blood mononuclear cells (PBMCs) from a subject diagnosed with or treated for an immune mediated disease, stimulating the PBMCs, identifying subpopulations of PBMCs, comparing data from the PBMC subpopulations to subject diagnosis and response to the therapeutic and selecting a signature marker profile related to a positive diagnosis or response. In one aspect, the PBMC stimulating agent of the method includes an anti-CD3/CD28 antibody, Tetanus toxoid peptides, PMA, ionomycin or other appropriately selected antigens, known to be contributors to the inflammatory process or any combination of the above. In another aspect, the PBMCs are isolated from a subject treated with a biologic agent. In one aspect, the immune mediated disease is rheumatoid arthritis (RA).

In a further aspect, the biologic therapeutic is an antibody which inhibits proteins and processes of the immune system and includes, but is not limited to, adalimumab, etanercept, infleximab, certolizumab, golimumab, anakinra, rituximab, abatacept, tocilizumab, muronomab, abciximab, daclizumab, basilimab, omalizumab, efalizumab, natalizumab, certolizumab pegol, usterkinumab, belimumab, clenoliximab, keliximab, priliximab, teneliximab, vapaliximab, ibalizumab, aselizumab, apolizumab, benralizumab, cedelizumab, certolizumab pegol, eculizumab, epratuzumab, erlizumab, fontolizumab, mepolizumab, ocrelizumab, pascolizumab, pexelizumab, reslizumab, rontalizumab, rovelizumab, ruplizumab, siplizumab, talizumab, teplizumab, tocilizumab, toralizumab, vedolizumab and visilizumab and any combination of the above. In yet another aspect, the therapeutic is an inhibitor of activated T cells; an inhibitor of B cells; an anti-inflammatory agent; a disease modifying agent; an analgesic agent or any combination thereof.

In a further aspect, the PBMC subpopulation identification includes FACS analysis. The markers identified by FACS analysis include, but is not limited to annexin V, B7-H1, B7-H3, B7-H4, B7-DC, B cells, memory B cells, CCR5, CCR6, CD1c, CD4, CD8, CD11c, CD14, CD16, CD19, CD25, CD 27, CD28, CD30, CD39, CD40, CD56, CD62L, CD69, CD80, CD86, CD103, CD107, CD123, CD127, CD141, CD161, CD200, CTLA-4, CXCR3, Fas, FasL, FoxP3, GARP, GATA-3, GITR, GranzymeA, GranzymeB, HLA-DR, ICOS, IL-1β, IL-2, IL-4, IL-6, IL-10, IL-12, IL-12R, IL17, IL-17A, IL-21, IL-23, INFγ, KOS, memory CD4 cells, monocyte cells, naïve CD4 cells, NK cells, NKT cells, OX40, PD-1, PD-LI/II, Perforin, RORC, STAT-3 phosphorylation, STAT-5 phosphorylation, surface LAP, T-bet, TCRgd, TGFβ, TNFα, T regulatory cells, T effector cells and any combination thereof.

In yet another aspect of the invention, the PBMC subpopulation identification includes a functional assay. The functional assay includes, but is not limited to, a proliferation assay, a suppression assay, confocal microscopy, Western blot, CTV staining, CFSE staining or propidium iodide (PI) assay. The confocal microscopy may further include determining the phosphorylation status of STAT-3 and STAT-5.

In another aspect, PBMC subpopulation identification is by PCR analysis. The PCR analysis includes but is not limited to FoxP3, GATA-3, Granzyme, ICOS, IκBα, IKK, IL-2, IL-4, IL-6, IL-10, IL-12, IL-12R, IL-17A, IL-21, IL-23, INFγ, NFκb pathway, p50, PD-1, Perforin, RelA, RORC, T-bet, TGFβ, TNFα and any combination thereof.

In a further aspect, the PBCM subpopulation includes but is not limited to CD4+, CD127+ and T effector cells; CD4+, CD127− and T regulatory cells; CD4+, CD127−, CD25low/neg and nT regulatory cells; CD4+, CD127−, CD25++ and nT regulatory cells; CD4+, CD127−, CD25++, PD-1+ and tT regulatory cells; CD14, CD19, CD11c and CD123; CD56+, CD3− and NK cells; CD8+ and T cells; CD4+, CXC3+, CCR6−, and Th1 cells; CD4+, CXC3, CCR6+, CD161+ and Th17 cells; CD4+, CXC3+, CCR6+, CD161+, Th1 cells and Th17 cells; CD8−, CD4+ and T cells; CD8+, CD4− and T cells; CD19+ and B cells; CD14+ and monocytes; CD4+, CD25high and T effector cells; CD4+, CD25− and T effector cells; and CD4−, CD8−, CD 19+ and B cells; and any combination thereof.

In another embodiment, the present invention provides a method of monitoring the course of a therapy using an immune mediated disease therapeutic. The method includes isolating PBMCs from a subject previously or currently treated for an immune mediated disease, stimulating the PBMCs, identifying subpopulations of PBMCs, comparing data from the PBMC subpopulations to a subject response to the therapeutic and selecting a signature marker profile related to a positive response to the therapeutic, thereby monitoring the course of therapy.

In a further embodiment, the present invention includes the use of an Immunomics platform to diagnose an immune mediated disease or predict response to an immune mediated disease therapeutic agent. The use includes isolating PBMCs from a subject diagnosed with or treated for an immune mediated disease, stimulating the PBMCs, identifying subpopulations of PBMCs, comparing data being comparing data from the PBMC subpopulations to subject diagnosis or response to the therapeutic and selecting a signature marker profile related to a positive diagnosis or response to the therapeutic agent.

In another embodiment, the present invention provides an epigenetic method to diagnose an immune mediated disease or to predict a response to an immune disease mediated therapeutic agent. The method includes isolating PBMCs from a subject diagnosed with or treated for an immune mediated disease, stimulating the PBMCs, identifying subpopulations of PBMCs, comparing data from the PBMC subpopulations to subject diagnosis or response to therapeutics and selecting a signature marker profile related to a positive diagnosis or response to the therapeutic. In one aspect the signature profile consists of genes or proteins which are up regulated or down regulated in response to therapy. In a further aspect the signature profile consists of methylation patterns of DNA or RNA in response to therapy.

In another aspect, the present invention provides for a protein or gene expression chip. In one aspect, the protein or gene expression chip contains one or more markers of the signature marker profile for predicting a response to a therapeutics.

### BRIEF DESCRIPTION OF THE DRAWINGS

FIG. 1 is a flow chart of the method to identify a signature marker profile for the response to RA therapy using the Immunomics platform.

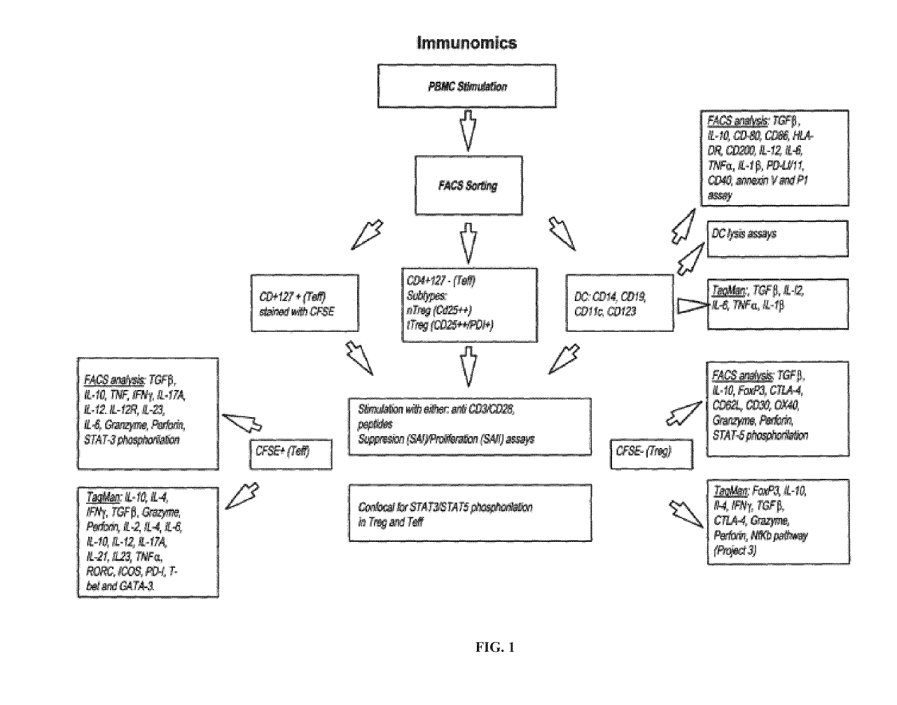

FIG. 2a through 2c are charts showing determination by TaqMan of the expression of IL-4, IL-10 and IL-17 from isolated T_{E}.

FIG. 3 is a chart showing the determination of suppression activity.

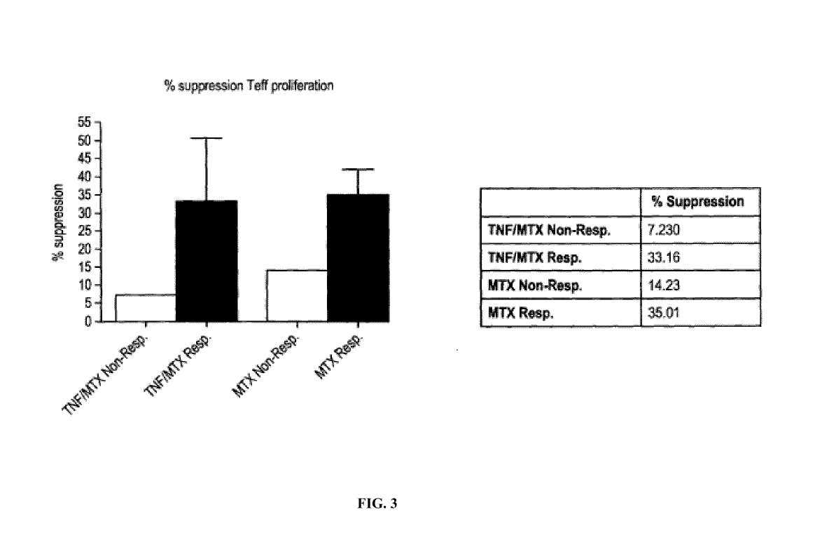

FIG. 4 is a heat map showing immunophenotyping of PBMCs from the TREAT trial were with a panel of markers using a 9-color FACSAria II.

FIG. 5 is a chart showing the staining for CD4, CD25, FoxP3, GITR, Granzyme B and CD69 on PBMCs from T₀ and T_{end}.

FIG. 6 is a heatmap based on p-value of a subset of factors analyzed using JIA samples.

FIG. 7 is a chart showing staining of T_{E} and T_{reg} cells at T0 and Tend by CD4, CD25, FoxP3, GITR, CCR5, CCR6, CXCR3, CD161, HLA-DR, GARP and surface LAP without manipulation.

FIG. 8 is a chart showing ROC curves using individual and a panel of parameters analyzed using JIA samples. The bold line indicates results from a multivariate analysis. All other lines are from univariate analyses.

FIG. 9 is a chart showing cluster analysis of multiple parameters analyzed using JIA samples.

FIG. 10 is a chart showing factorial analysis using two parameters analyzed using JIA samples.

FIG. 12 is a PCA graph visually demonstrates that clinical manifestations alone are unable to predict or confirm treatment response.

FIG. 13 is shows complete dataset of predictive (T0) and confirmatory variables (Tend).

FIG. 14 shows individual variables further identified from T0.

FIG. 15 shows a visual representation by Principal Component Analysis (PCA), showing how the populations of ID and NO ID patients are separated by our biomarker cluster.

FIG. 16 shows clusters predicted ID and NO ID patients with an overall accuracy of 87.5%.

FIG. 17 is shows significant or nearly significant parameters of CD4+ T cells at end of study (Tend, Confirmatory).

FIG. 18 is visualization by a PCA representation of how the populations of ID and NO ID patients separated by the clusters versus clinical parameters.

FIG. 19 shows clustering parameters improved the accuracy of classification, segregating ID and NO ID patients with an overall accuracy of 87.5%

### DETAILED DESCRIPTION OF THE INVENTION

The present invention is based in part on the identification of a marker profile of immune variables to diagnose an immune mediated disease or predict a subject's response to an immune mediated disease therapeutic agent. Additionally, the present invention provides methods for the prediction of a subject's response to an immune mediated disease therapeutic agent and the monitoring of a course of treatment with such a therapeutic agent. Furthermore, the present invention provides for protein and gene expression chips containing the signature marker profile.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “immune mediated disease” refers to conditions which result from abnormal activity of the body's immune system. Immune mediated diseases can include but are not limited to immunodeficiency diseases, autoimmune diseases and hypersensitivity diseases. Autoimmune diseases are the result of the immune system attacking the body instead of pathogens. Immunodeficiency diseases occur when part of the immune system is not functioning. Autoimmune diseases are the result of the immune system attacking the body instead of pathogens. Hypersensitivity diseases occur when the immune system over reacts and results in damage to the body.

Immunodeficiency diseases occur when part of the immune system is not functioning properly. Primary immunodeficiency occurs when a person is born lacking a part of the immune system which impairs the immune processes from functioning normally. Acquired immunodeficiency occurs when the body stops producing a component of the immune system. Treatments for immunodeficiency include antibiotics, immunoglobulin therapy, gamma interferon therapy, stem cell transplantation and growth factors.

Hypersensitivity occurs when the immune system over reacts and results in damage to the body. Typically there are four types of hypersensitivity reactions; IgE antibody reaction, Cytokine reaction, immune complex reaction and delayed reaction. Treatments for hypersensitivity include antihistamine medications, corticosteroids, DMARDS, epinephrine injection, immunosuppressive agents, leukotriene receptor antagonists, mast cell stabilizers, and NSAIDS.

Autoimmune diseases are the result of the immune system attacking the body instead of pathogens. There are many types of autoimmune disorders and each uses a different treatment. In general, treatment is directed toward symptom relief, organ preservation and targeting disease mechanisms. Some of the possible treatments include thyroid supplements, insulin, blood transfusion, immunosuppressive agents, NSAIDS, and interferon beta.

Immune mediated diseases include, but are not limited to: allergic bronchopulmonary aspergillosis; allergic rhinitis, autoimmune hemolytic anemia; acanthosis nigricans; allergic contact dermatitis; Addison's disease; atopic dermatitis; alopecia greata; alopecia universalis; amyloidosis; anaphylactoid purpura; anaphylactoid reaction; aplastic anemia; angioedema, hereditary; angioedema, idiopathic; ankylosing spondylitis; arteritis, cranial; arteritis, giant cell; arteritis, Takayasu's; arteritis, temporal; asthma; ataxia-telangiectasia; autoimmune oophoritis; autoimmune orchitis; autoimmune polyendocrine failure; Behcet's disease; Berger's disease; Buerger's disease; bronchitis; bullous pemphigus; candidiasis, chronic mucocutaneous; Caplan's syndrome; post-myocardial infarction syndrome; post-pericardiotomy syndrome; carditis; celiac sprue; Chagas's disease; Chediak-Higashi syndrome; Churg-Strauss disease; Cogan's syndrome; cold agglutinin disease; CREST syndrome; Crohn's disease; cryoglobulinemia; cryptogenic fibrosing alveolitis; dermatitis herpetifomis; dermatomyositis; diabetes mellitus; Diamond-Blackfan syndrome; DiGeorge syndrome; discoid lupus erythematosus; eosinophilic fasciitis; episcleritis; drythema elevatum diutinum; erythema marginatum; erythema multiforme; erythema nodosum; Familial Mediterranean fever; Felty's syndrome; pulmonary fibrosis; glomerulonephritis, anaphylactoid; glomerulonephritis, autoimmune; glomerulonephritis, post-streptococcal; glomerulonephritis, post-transplantation; glomerulopathy, membranous; Goodpasture's syndrome; granulocytopenia, immune-mediated; granuloma annulare; granulomatosis, allergic; granulomatous myositis; Grave's disease; Hashimoto's thyroiditis; hemolytic disease of the newborn; hemochromatosis, idiopathic; Henoch-Schoenlein purpura; hepatitis, chronic active and chronic progressive; histiocytosis X; hypereosinophilic syndrome; rheumatoid arthritis (Juvenile chronic arthritis); Kawasaki's disease; keratitis; keratoconjunctivitis sicca; Landry-Guillain-Barre-Strohl syndrome; leprosy, lepromatous; Loeffler's syndrome; lupus; Lyell's syndrome; lyme disease; lymphomatoid granulomatosis; mastocytosis, systemic; mixed connective tissue disease; mononeuritis multiplex; Muckle-Wells syndrome; mucocutaneous lymph node syndrome; mucocutaneous lymph node syndrome; multicentric reticulohistiocytosis; multiple sclerosis; myasthenia gravis; mycosis fungoides; necrotizing vasculitis, systemic; nephrotic syndrome; overlap syndrome; panniculitis; paroxysmal cold hemoglobinuria; paroxysmal nocturnal hemoglobinuria; pemphigoid; pemphigus pemphigus erythematosus; pemphigus foliaceus; pemphigus vulgaris; pigeon breeder's disease; pneumonitis, hypersensitivity; polyarteritis nodosa; polymyalgia rheumatic; polymyositis; polyneuritis, idiopathic; portuguese familial polyneuropathies; pre-eclampsia/eclampsia; primary biliary cirrhosis; progressive systemic sclerosis (scleroderma); psoriasis; psoriatic arthritis; pulmonary alveolar proteinosis; pulmonary fibrosis, Raynaud's phenomenon/syndrome; Reidel's thyroiditis; Reiter's syndrome, relapsing polychondritis; rheumatic fever; rheumatoid arthritis (RA); sarcoidosis; scleritis; sclerosing cholangitis; serum sickness; Sezary syndrome; Sjogren's syndrome; Stevens-Johnson syndrome; Still's disease; subacute sclerosing panencephalitis; sympathetic ophthalmia; systemic lupus erythematosus; transplant rejection; ulcerative colitis; undifferentiated connective tissue disease; urticaria, chronic; urticaria, cold; uveitis; vitiligo; Weber-Christian disease; Wegener's granulomatosis, or Wiskott-Aldrich syndrome. Immune mediated disease may also include transplant organ rejection such as heart, kidney, liver, skin, pancreatic islet cells or bone marrow rejection. Immune mediated disease may also include graft-vs-host disease in a subject.

Additionally, immune mediated diseases may include pulmonary fibrosis or fibrotic disease. Some examples of pulmonary fibrosis include: pulmonary fibrosis secondary to adult respiratory distress syndrome, drug-induced pulmonary fibrosis, idiopathic pulmonary fibrosis, or hypersensitivity pneumonitis. Some examples of fibrotic diseases include: Hepatitis-C; Hepatitis-B; cirrhosis; cirrhosis of the liver secondary to a toxic insult; cirrhosis of the liver secondary to drugs; cirrhosis of the liver secondary to a viral infection; and cirrhosis of the liver secondary to an autoimmune disease.

Further, immune mediated disease may include, but are not limited to, gastrointestinal disease including: esophageal dysmotility, inflammatory bowel disease (including Crohn's disease and ulcerative colitis), gastritis, collagenous colitis (including lymphocytic colitis and microscopic colitis), coeliac disease (also called gluten enteropathy, coeliac sprue, or gluten intolerance), and scleroderma. In another aspect immune mediated disease may include: atherosclerosis, renal artery disease, lymphedema, ischemic disorders, reperfusion injury, collagen vascular/immune complex diseases such as systemic lupus erythematosis or cryoglobulinemia.

Further, autoimmune diseases may include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitis); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia, Myasthenia gravis, systemic lupus erythematosus, Graves' disease, idiopathic thrombocytopenia purpura, hemolytic anemia, diabetes mellitus, inflammatory bowel disease, Crohn's disease, multiple sclerosis, psoriasis, drug-induced autoimmune diseases, or drug-induced lupus. In certain embodiments, the autoimmune response is associated with or derived from systemic lupus erythematosus, infectious disease, Reiter's syndrome, spondylarthritis, Lyme disease, HIV infection, syphilis, or tuberculosis etc.

Additionally, immune mediated disease also includes traumatic brain injury and depression.

As used herein, the terms “rheumatoid arthritis” or “RA” refers to an autoimmune disease which primarily affects joints. RA includes, but is not limited to, adult RA, juvenile iodopathic arthritis, juvenile rheumatoid arthritis and juvenile chronic arthritis.

RA is an example of an autoimmune mediated disease and is incurable, once bone or joint damage occurs it is usually irreversible. It is therefore, important that RA treatment begin soon after the onset of symptoms to delay or halt disease progression and prevent irreversible damage.

As used herein, the term “an immune mediated disease therapeutic” or “immune mediated disease therapeutic agent” refers to any therapeutic agent used to treat an immune mediated disease.

DMARDs are a broad class of agents which are know as disease modifying anti-rheumatic drugs, which are used in a variety of immune mediated diseases. DMARDS include azathioprine, ciclosporin, D-penicillamine. Gold salts, hydroxychloroquine, leflunomide, methotrexate, minocycline, sulfasalazine and cyclophosphamide. Treatments encompassing combinations of DMARDS are common.

Analgesics and anti-inflammatory agents are also frequently used in treating immune mediated diseases. Typical analgesics include acetaminophen, opiates, diproqualone, and topical lidocaine. Typical anti-inflammatory agents include glucocorticoids and non-steroidal anti-inflammatory agents (NSAIDS). Glucocorticoids include hydrocortisone, prednisone, prednisolone, methylprednisolone, dexamethasone, betamethasone, triamcinolone, beclometasone, fludrocortisone acetate, deoxycorticosterone acetate, and aldosterone. Typical NSAIDS include ibuprofen, maproxen, meloxicam, etodolac, nabumetone, sulindac, tolementin, choline magnesium salicylate, diclofenac, diflusinal, indomethicin, ketoprofen, oxaprozin and piroxicam.

Biologic agents are medicinal products such as a vaccine, blood or blood component, allergenic, somatic cell, gene therapy, tissue, recombinant therapeutic protein, or living cells that are used as therapeutics. Unlike other drugs which are chemically synthesized, biologic agents are made through biologic processes. For example, therapeutic antibodies are produced in live cells and secreted into the surrounding media. Biologic agents have been very effective in alleviating symptoms of arthritis. Many of these agents affect the immune system and t have some significant side effects and are therefore usually used only after other treatments have failed. Side effects that can occur with these agents include injection site reactions (rash, burning), tuberculosis, bacterial infections, allergic reactions and progressive multifocal leukoencephalopathy, which can be fatal. It is therefore imperative that patients on an immune mediated disease therapeutic agent which is a biologic be monitored closely and ideally patients would be screened prior to treatment to determine which biologic agent would be effective.

Biologic agents used as immune mediated disease therapeutics inhibit various different immune system related proteins and processes. Adalimumab, etanercept, infleximab, certolizumab, and golimumab inhibit TNF. Anakinra targets Il-1. Rituximab targets CD20 on B cells. Abatacept acts as a T cell costimulation blocker. Tocilizumab targets IL-6. Other examples of biologic immune mediated disease therapeutics include, but is not limited to, muronomab, abciximab, daclizumab, basilimab, omalizumab, efalizumab, natalizumab, certolizumab pegol, usterkinumab, belimumab, clenoliximab, keliximab, priliximab, teneliximab, vapaliximab, ibalizumab, aselizumab, apolizumab, benralizumab, cedelizumab, certolizumab pegol, eculizumab, epratuzumab, erlizumab, fontolizumab, mepolizumab, ocrelizumab, pascolizumab, pexelizumab, reslizumab, rontalizumab, rovelizumab, ruplizumab, siplizumab, talizumab, teplizumab, tocilizumab, toralizumab, vedolizumab and visilizumab.

In one embodiment, the present invention provides a method for diagnosis of an immune mediated disease or predicting a response to an immune mediated disease therapeutic agent. The method includes isolating PBMCs from a subject diagnosed with or treated for an immune mediated disease, stimulating the PBMCs, determining subpopulations of PBMCs, comparing data from the PBMC subpopulations to a subject diagnosis or response to therapeutic agent and selecting a marker profile related to a positive diagnosis or response to therapeutic.

As used herein, the term “PBMC” refers to peripheral blood mononuclear cell. A PBMC is any blood cell having a round nucleus. These cells are critical components of the immune system to fight infection. PBMCs include lymphocytes, monocytes and macrophages. Lymphocytes include T cells, B cells and natural killer (NK) cells. PBMCs can be stimulated so that T cells proliferate. Agents that can stimulate PBMCs and therefore T cell proliferation, include anti CD3/CD28 antibody, Tetanus toxoid peptides, PMA, ionomycin or other appropriately selected antigens, known to be contributors to the inflammatory process or any combination of the above.

T cells are integral to cell mediated immunity and all express CD4. There are several types of T cells: helper T cells (T_{H} cells), cytotoxic T cells (T_{C} cells), memory T cells, regulatory T cells (T_{reg} cells), effector T cells (T_{E} cells) and natural killer cells (NKT cells). T_{H} cells assist other T cells in immunologic presses, such as maturation of B cells and activation of T_{C} cells. T_{H} cells are activated by MCH class II molecules expressed on the surface of antigen presenting cells (APCs). T_{reg} cells are responsible for shutting down T cell mediated immunity and express CD4, CD25 and FoxP3. T_{E} cells contribute to the fundamental reactions of adaptive immunity.

PBCMs are isolated from the blood typically using ficoll which traps monocytes and lymphocytes in a buffy coat which forms underneath plasma in a tube. Another way to isolate PBMCs is to extract the cells from whole blood using hypotonic lysis which will preferentially lyse red blood cells.

An aspect of the present invention includes the identification of a subpopulation of PBMCs from patients that had a positive diagnosis for an immune mediated disease or positive response to an immune mediated disease therapeutic agent. This subpopulation of PBMCs will have differences in immune variables compared to those patients that didn't have a positive diagnosis or response to therapy. This difference in immune variables provides a way to identify patients who will also have a positive diagnosis or response to the therapeutic and is therefore important to help with clinical decisions. Given the severity of the side effects associate with treatment using some immune mediated disease therapeutics, such information is invaluable. There are several methods that can be used alone or in combination to identify this subpopulation including FACS analysis, functional assays and PCR analysis. Subpopulations of the instant invention may include, but are not limited to, CD4⁺, CD 127⁺ and T effector cells; CD4⁺, CD 127⁻ and T regulatory cells; CD4⁺, CD 127⁻, CD25^{low/neg} and nT regulatory cells; CD4⁺, CD 127⁻, CD25⁺⁺ and nT regulatory cells; CD4⁺, CD127⁻, CD25⁺⁺, PD-1⁺ and tT regulatory cells; CD14, CD19, CD11c and CD123; CD56⁺, CD3⁻ and NK cells; CD8⁺ and T cells; CD4⁺, CXC3⁺, CCR6⁻, and Th1 cells; CD4⁺, CXC3, CCR6⁺, CD161⁺ and Th17 cells; CD4⁺, CXC3⁺, CCR6⁺, CD161⁺, Th1 cells and Th17 cells; CD8⁻, CD4⁺ and T cells; CD8⁺, CD4⁻ and T cells; CD19⁺ and B cells; CD14⁺ and monocytes; CD4⁺, CD25^{high} and T effector cells; CD4⁺, CD25⁻ and T effector cells; and CD4⁻, CD8⁻, CD 19⁺ and B cells; and any combination thereof.

In one aspect of the invention, FACS analysis may be used to sort cells based upon the specific light scattering and fluorescent characteristics of each cell. Cells are fluorescently labeled for different characteristics (such as expression of certain proteins) and the FACS apparatus can separate the cells into distinct populations. In the present invention FACS analysis may be used for the isolation of different types of T cells through specific cell surface markers or as a means of identifying the subpopulation of PBMCs from patients that responded to therapy based on a unique combination of cell surface markers. For example the cell surface markers may include, but are not limited to, annexin V, B7-H1, B7-H3, B7-H4, B7-DC, B cells, memory B cells, CCR5, CCR6, CD1c, CD4, CD8, CD11c, CD14, CD16, CD19, CD25, CD 27, CD28, CD30, CD39, CD40, CD56, CD62L, CD69, CD80, CD86, CD103, CD107, CD123, CD127, CD141, CD161, CD200, CTLA-4, CXCR3, Fas, FasL, FoxP3, GARP, GATA-3, GITR, GranzymeA, GranzymeB, HLA-DR, ICOS, IL-1β, IL-2, IL-4, IL-6, IL-10, IL-12, IL-12R, IL17, IL-17A, IL-21, IL-23, INFγ, KOS, memory CD4 cells, monocyte cells, naïve CD4 cells, NK cells, NKT cells, OX40, PD-1, PD-LI/II, Perforin, RORC, STAT-3 phosphorylation, STAT-5 phosphorylation, surface LAP, T-bet, TCR_{gd}, TGFβ, TNFα, T regulatory cells, T effector cells and any combination thereof.

In another aspect of the invention, the identification of the subpopulation of PBMCs may include the use of functional assays. Functional assays are assays that measure an activity of the cell. Functional assays include proliferation assays and suppression assays which measure the growth or lack thereof of a cell population. Other functional assays include staining with CTV (Cell Trace Violet) to identify T_{reg} cells and carboxyfluorescein diacetate succinimidyl ester (CFSE) to identify T_{E} cells. Another functional assay is confocal microscopy which may be used to determine the phosphorylation status of STAT3 and STATS. Additionally, western blot analysis can be used to detect the presence or absence of proteins.

In a further aspect, PCR analysis may also be used for the identification of the subpopulation of PBMCs. PCR analysis can be used to identify genes that are expressed and to quantify and identify changes in expression of genes. PCR markers of the present invention include, but are not limited to, FoxP3, GATA-3, Granzyme, ICOS, IκBα, IKK, IL-2, IL-4, IL-6, IL-10, IL-12, IL-12R, IL-17A, IL-21, IL-23, INFγ, NFκb pathway, p50, PD-1, Perforin, RelA, RORC, T-bet, TGFβ, TNFα and any combination thereof.

As used herein, the term “signature marker profile” refers to a set of immune variables identified from PBMCs of patients which have been diagnosed with an immune mediated disease or have responded to an immune mediated disease therapeutic agent. This signature marker profile may then be used to diagnose an immune mediated disease or to screen patients and predict whether these patients would have a positive response to an immune mediated disease therapeutic agent. Pre-screening patients would aid in clinical decisions and reduce any unnecessary side effects by ensuring patients are only given therapeutics for which they are predicted to have a positive response. The signature marker profile may also be used to monitor patient currently undergoing therapy to ensure the immune mediated disease therapeutic agent is working. The signature profile may vary depending on the specific therapeutic agent which is used.

In another embodiment, the present invention provides a method of monitoring the course of a therapy using an immune mediated disease therapeutic agent. The method includes isolating PBMCs from a subject diagnosed with or treated for an immune mediated disease, stimulating the PBMCs, determining subpopulations of PBMCs, comparing data from the PBMC subpopulations to a subject response to the therapeutic agent and selecting a marker profile related to a positive response to the therapeutic agent.

In a further embodiment, the present invention provides a method of predicting response to an immune mediated disease therapeutic agent. The method including obtaining a blood sample from a subject in need of therapy, analyzing the blood sample for the signature marker profile for positive response to the therapeutic agent and identifying the profile markers present in the blood sample. In one aspect of the method, the blood sample is analyzed by methods including a protein expression chip; a gene expression chip, protein expression, detection of phosphorylation, phosphorescence or luminescence detection, immunobeads or a combination thereof.

In one aspect, the present invention provides a method for the diagnosis of traumatic brain injury. The method including obtaining a blood sample from a subject in need of such a diagnosis, analyzing the blood sample for the signature marker profile identified for traumatic brain injury and identifying the profile markers present in the blood sample. In one aspect of the method, the blood sample is analyzed by methods including a protein expression chip; a gene expression chip, protein expression, detection of phosphorylation, phosphorescence or luminescence detection, immunobeads or a combination thereof.

In another aspect, the present invention provides a method to distinguish between Type I and Type II diabetes. The method including obtaining a blood sample from a subject suspected of having Type I or Type II diabetes, analyzing the blood sample for the signature marker profile for Type I and Type II diabetes and identifying the profile markers present in the blood sample. In one aspect of the method, the blood sample is analyzed by methods including a protein expression chip; a gene expression chip, protein expression, detection of phosphorylation, phosphorescence or luminescence detection, immunobeads or a combination thereof.

In a further aspect, the present invention provides a method to diagnosis and treat depression. The method including obtaining a blood sample from a subject in need of a diagnosis or therapy for depression, analyzing the blood sample for the signature marker profile identified for depression and identifying the profile markers present in the blood sample. In one aspect of the method, the blood sample is analyzed by methods including a protein expression chip; a gene expression chip, protein expression, detection of phosphorylation, phosphorescence or luminescence detection, immunobeads or a combination thereof.

In another aspect, the present invention provides for a protein or gene expression chip containing the signature marker profile. Protein and gene expression chips are used to detect and measure levels of either proteins, DNA or RNA. The protein or gene expression chips of the present invention may be used to confirm a diagnosis and/or predict which patients will respond to an immune mediated disease therapeutic and/or to monitor a patient undergoing a course of treatment with such a therapeutic.

In a further aspect, the present invention includes the use of an Immunomics platform to support diagnose an immune mediated disease or predict response to an immune mediated disease therapeutic agent. The use includes isolating PBMCs from a subject treated for an immune mediated disease, stimulating the PBMCs, determining subpopulations of PBMCs, comparing data comparing data from the PBMC subpopulations to a subject diagnosis or response to the therapeutic and selecting a marker profile related to a positive diagnosis or response to the therapeutic.

In another embodiment of the present invention Immunomics platform is used to diagnose subjects with immune mediated disease and to predict which subjects will respond to an immune mediated disease therapeutic agent and/or to monitor a patient undergoing a course of treatment with such a therapeutic agent. The Immunomics platform is the integration of several technologies into a single platform. These technologies include, but are not limited to, FACS analysis, functional assays, PCR analysis, a protein expression chip; a gene expression chip, protein expression, detection of phosphorylation, phosphorescence or luminescence detection, immunobeads or a combination thereof.

In another embodiment, the present invention provides an epigenetic method to support a diagnosis diagnose an immune mediated disease or for predicting a response to an immune mediated disease therapeutic agent. The method includes isolating proliferating blood mononuclear cells (PBMCs) from a subject diagnosed with or treated for an immune mediated disease, stimulating the PBMCs, identifying subpopulations of PBMCs, comparing data from the PBMC subpopulations to subject diagnosis or response to therapeutic agent and selecting a signature marker profile related to a positive diagnosis or response to the therapeutic. In one aspect the signature profile consists of genes or proteins which are up regulated or down regulated in response to therapy. In a further aspect the signature profile consists of methylation patterns of DNA or RNA in response to therapy.

In another aspect, the present invention provides for a protein or gene expression chip. In one aspect, the protein or gene expression chip contains the signature marker profile for predicting response to an immune mediated disease therapeutic agent. In one aspect the signature profile consists of genes or proteins which are up regulated or down regulated in response to therapy. In a further aspect the signature profile consists of methylation patterns of DNA or RNA in response to therapy.

The following examples are intended to illustrate but not limit the invention.

### Example 1

The aim of this study is to identify clusters of immune pathways characteristic of responsiveness that are specific to treatment. The data below pertain to a preliminary study conducted in patients with RA, (n=8), who were first treated with methotrexate (MTX) and stratified based on clinical responsiveness based on Disease Activity Score of 3.2 (DAS3.2). Non-responders (DAS>5.1) were given an anti-TNFα antibody (herein TNF or ETN) in addition to MTX. We show as summary a color coded table. We clustered individual immune functions according to cut off values identified in preliminary studies (25% change compared with baseline at active disease). The Table 1 depicts the differences for patients who are all responders and therefore otherwise clinically indistinguishable (DAS 23.2).

PBMCs from each patient were incubated for 48 hours with plate bound anti-CD3/CD28. Non-adherent cells were collected to enrich T cells by pan T-cell MACS. T cells were FACS sorted on CD4+CD127+ (T_{E}) and CD4+CD25+CD127− (nTreg), and CD4+CD25+PD1+CD127− (PD1+ Treg). Aliquots of all sorted cells were collected for TaqMan. The remainder of the APCs and TE were cultured with isolated Treg. CFSE was added to the TE cells and cultured for a total of 4 days with anti-CD3/CD28.

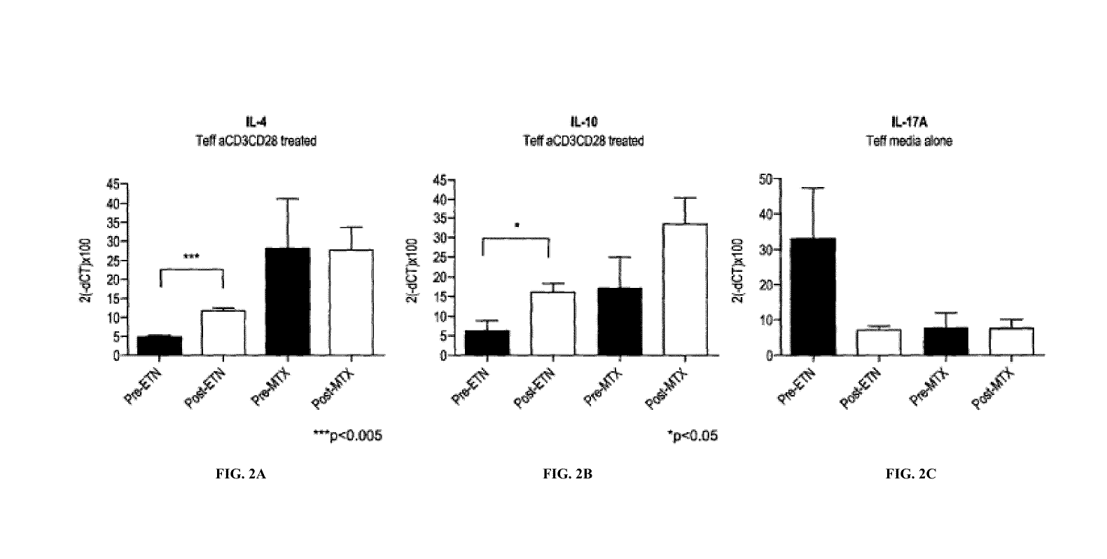

The treatment-induced changes in effector T cells as demonstrated by IL-4 and IL-10 levels which were significantly restored in MTX failures after successful combination therapy (FIG. 2). Basal levels are profoundly different between the two groups at TO. This applies also to the pro-inflammatory cytokine IL-17. Levels of IL-17 were elevated at TO in MTX failures; whereas successful ETN/MTX treatment leads to a reduction of IL-17 expression.

Regulatory T cell suppression is restored by treatment. In both MTX-alone and anti-TNFα antibody/MTX treatment, suppressive capability of Treg to suppress effector proliferation is enhanced when clinical response is seen.

Taken together, these preliminary studies, which need to be sufficiently powered and then validated, suggest the existence of a circuit of active immune tolerization, which seems relevant to clinical improvement. Importantly, this preliminary work also suggests the presence of differences in immune function between the group of MTX failures and MTX.

TABLE 1
Visual representation of levels of gene expression for T_{reg}, T_{E} and APC pertaining to clinical responders to MTX-
ETN (labeled TNF) and to MTX alone. Values are compared to initiation of the treatment and represent the percent
change in expression of a given gene as measured by Taqman. Unpaired t test was used for statistical analysis.

| | APC | | | | Tₑ Cells | | | |
|---|---|---|---|---|---|---|---|---|
| | | up or down | | up or down | | up or down | | up or down |
| | TNF resp | regulated | MTX resp | regulated | TNF resp | regulated | MTX resp | regulated |
| Tbet | | | | | −5.2% | | −17.1% | |
| GATA-3 | | | | | 25.9% | up regulated | −21.6% | |
| IL-10 | −0.05% | | +28.6% | up regulated | +28.6% | up regulated | +15.2% | no change |
| IL-17A | | | | | −25.1% | down | +0.3% | no change |
| | | | | | | regulated | | |
| IL-21 | | NA | | NA | −71.3% | down | | NA |
| | | | | | | regulated | | |
| TNFα | | NA | | NA | −401% | | 2.3% | |
| INFγ | +40.7% | up regulated | +219.9% | up regulated | 41.0% | down | +1.7% | |
| | | | | | | regulated | | |
| TGFβ | +18.0% | | +31.1% | up regulated | 6.3% | | +150.8% | up regulated |
| PD-1 | | | | | 24.1% | | +5.6% | |
| FAS-L | | | | | +29.7% | up regulated | 20.5% | |
| ICOS | | | | | −23.8% | | −7.2% | |
| B7-H1 | +94.0% | up regulated | +106.4% | up regulated | | | | |
| B7-H2 | +1681.2% | up regulated | +98.4% | up regulated | | | | |
| B7-H3 | 70.0% | up regulated | | NA | | | | |
| B7-H4 | +1014.2% | up regulated | +118.6% | up regulated | | | | |
| B7-DC | | NA | | NA | | | | |
| CD80 | +66.7% | up regulated | | NA | | | | |
| CD86 | +16.7% | | | NA | | | | |
| FAS-L | −92.0% | down | +83.1% | up regulated | | | | |
| | | regulated | | | | | | |
| | nTreg | | | | PD1 + Treg | | | |
| | TNF | | MTX | | TNF | | MTX | |
| FoxP3 | +22.7% | no change | 439.0% | up regulated | −73.3% | down | +124.1% | up regulated |
| | | | | | | regulated | | |
| PD1 | +54.4% | up regulated | +474.2% | up regulated | −80.0% | down | 290.3% | up regulated |
| | | | | | | regulated | | |
| CTLA4 | 20.8% | no change | +684.0% | up regulated | −67.6% | down | +161.0% | up regulated |
| | | | | | | regulated | | |
| Granzyme | −37.7% | down | +393.7% | up regulated | −29.9% | down | +213.8% | up regulated |
| B | | regulated | | | | regulated | | |
| Perforin | +96.6% | up regulated | | NA | +91.3% | up regulated | +288.7% | up regulated |
| TGFβ | +16.1% | no change | +69.3% | up regulated | −2.6% | no change | +254.4% | up regulated |
| IL-10 | +9.4% | no change | | NA | −78.8% | down | 168.8% | up regulated |
| | | | | | | regulated | | |
| INFγ | −41.1% | down | +99.6% | up regulated | +35.5% | up regulated | +355.0% | up regulated |
| | | regulated | | | | | | |

### Example 2

The TREAT study is a recently concluded clinical trial, which tested the difference between two treatment (MTX+anti-TNFα antibody+prednisone+NSAIDS and MTX+prednisone+NSAIDS) arms in patients with active JIA who achieved inactive disease (ID). Samples from most patients were collected and stored frozen at two time points: Initiation of treatment (T₀) and end (T_{end}). The data below are from a set of 10 patients: 7 with ID and 3 with no ID in the same treatment arm.

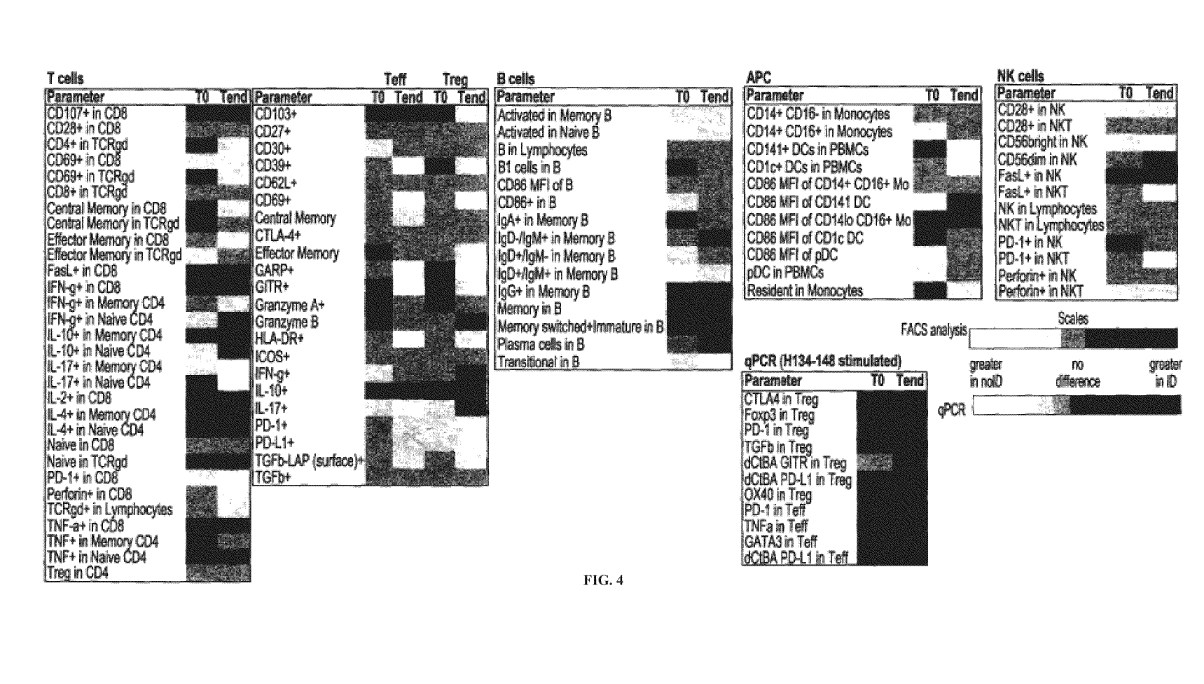

PBMCs from the TREAT trial were thawed and a fraction immunophenotyped with a panel of markers using a 9-color FACSAria II. Any residual PBMCs were sorted for CD4⁺CD25^{low/neg}CD127⁺ T_{E}, CD4⁺CD25^{high}CD127^{low/neg} (T_{reg}), CD19+ B cells, CD56+CD3− NK cells and CD8+ T cells for functional assays. T_{E} and T_{reg} were differentially labeled with vital dyes (CFSE and CTV respectively) to discriminate them, then stimulated with H134-148 peptide in the presence of autologous APC. After 2 days, T_{E} and T_{reg} were sorted for qPCR analysis of gene expression. CD8+ T cells were stimulated for 6 hours with anti-CD3 crosslinked by P815 cells in the presence of CD107a antibody, monensin and brefeldin A. At the end of the experiment, the percentage of degranulating CD8+ T cells was assessed by the expression of CD107a. In addition, an intracellular staining for IFN-γ, TNF-α and IL-2 was performed on those cells. Other functional assays, not shown here, include proliferation and suppression assays of CD4+ T cells, activation of B cells via B cell-receptor, CD40 and/or Toll-like receptors to determine proliferation and cytokine production, and NK cell degranulation in response to K562 targets. Data were stratified by time point and disease activity. Differences in each parameter between inactive or not inactive disease within a time point were normalized then color-coded in a heat map (FIG. 4). The heat map represents changes in each parameter between patients who ended therapy with inactive disease versus those who still showed active disease (FIG. 4). Changes are calculated as differences of values normalized in a 0 to 100% scale (where 100% is the maximum value in the whole dataset for each parameter) for flow cytometry data or as Log 2 of relative expression for qPCR data.

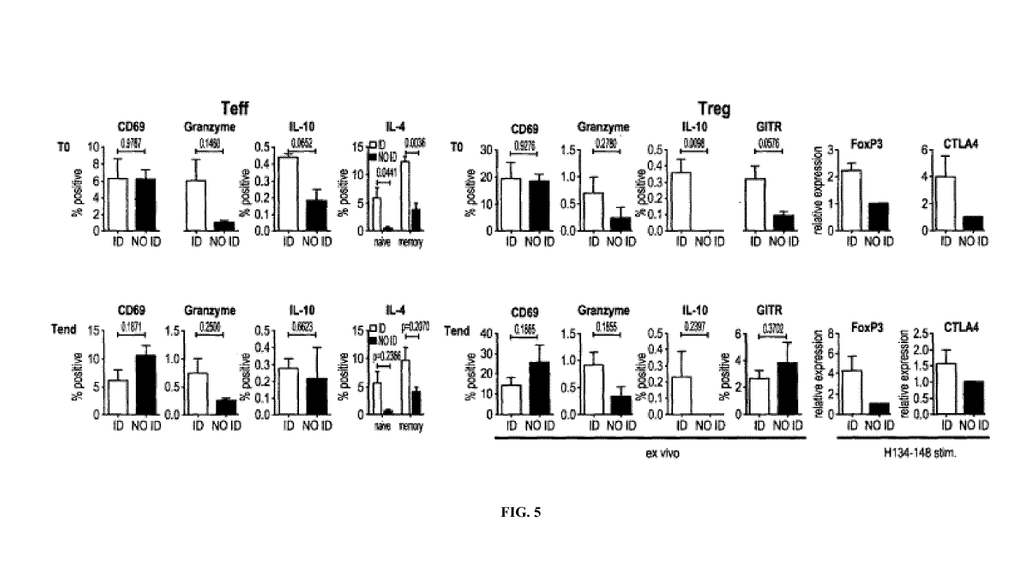

Thawed PBMC from T₀ and T_{end} were stained for CD4, CD25, FoxP3, GITR, Granzyme B and CD69 without manipulation (n=6 to 7 for ID, n=3 for NO ID). Analyses were performed on gated CD4+CD25highFoxP3+ (T_{reg}) or CD4⁺CD25^{low/neg} (T_{E}) cells. A portion of the cells were stimulated with PMA/ionomycin for 6 hr (in the presence of brefeldin A for the last 4 hr) before staining for intracellular IL-10 and IL-4 (n=2 to 3 for ID, n=3 for NO ID). In functional assays, PBMC were sorted into CD4⁺CD25⁻CD127⁺ T_{E} and CD4⁺CD25^{high}CD127^{low/neg} T_{reg}. T_{E} were labeled with CFSE, T_{reg} with CTV, a CFSE analog. T cells were then co-cultured with APC presenting H134-148. After 2 days, cells were sorted based on differential labeling (CTV for T_{reg} and CFSE for TT_{E}) to perform qPCR for CTLA-4 and FoxP3 expression (n=2 for ID, n=1 for NO ID), shown in relative scale using the “NO ID” condition as reference (FIG. 5). ID=PBMC from patients who reached inactive disease state at the end of the TREAT clinical trial; NO ID=PBMC from patients who did not reach inactive disease state at the end of the trial.

Heat map of immune functions tested in TREAT evidences clustering associated with responsiveness to therapy. For visualization purposes only, we organized the 132 immune variables tested into a heat map (FIG. 4). Even within the limited sample size tested to date, the relevance and feasibility of the approach in identifying individual and clustered immune functions associated with responsiveness to therapy are evident

From the heat map shown above (FIG. 4) a cluster of phenotypical and functional markers that characterize T_{E} and T_{reg} were identified. Importantly, these data are obtained both ex vivo (i.e. directly by FACS from unmanipulated PBMC, as in SA 1) and upon stimulation with H134-148, an HSP-derived peptide (as in SA 2). FIG. 5 shows that baseline (T₀) expression of IL-10, Granzyme B, and GITR, and CTLA-4 (upon stimulation in vitro with H134-148) are elevated in patients who will achieve ID. T_{E} function appears skewed toward a more tolerogenic phenotype at T₀ in future ID patients as well. At T_{end}, T_{reg} in patients with ID showed higher values of IL-10, Granzyme and FoxP3 (upon stimulation with H134-148). T_{E} in ID patients showed a lower proportion of activated cells (CD69+) and higher values of Granzyme B and IL-4.

The preliminary data shown provide proof of feasibility with respect to 1) collection, preparation, shipping and processing of high quality biologic samples as part of a clinical trial performed in pediatric rheumatology sites; 2) adequacy of cell numbers in the biologic samples to perform the assays; 3) adequate power to perform the biologic assays; 4) most importantly these data give a clear indication that distilling a cluster of immune responses relevant to a given clinical condition is feasible.

### Example 3

Patient samples are collected from both a JIA study and a RA study. Both studies have two treatment arms: MTX+anti-TNFα antibody+prednisone+NSAIDS and MTX+prednisone+NSAIDS. Samples are collected from time points T₀ and T_{end} of both arms of both studies. Patients are stratified into groups based on response to treatment. The patient samples are analyzed using a gene chip for example. Genes which are expressed and genes which are not expressed are identified for the groups that responded to treatment. A signature marker profile is then identified based on the gene expression pattern and/or the DNA or RNA methylation pattern.

### Example 4

Frozen samples were collected from JIA patients a clinical trial testing MTX+anti TNFα antibody+prednisone versus MTX alone (the TREAT study). Samples were collected from 10 patients who achieved inactive disease (ID) and 9 patients who did not achieve ID at initiation of treatment (T₀) and end of treatment (T_{end}).

Several immunologic variables were captured in a semi-high throughput fashion using the Immunonics platform.

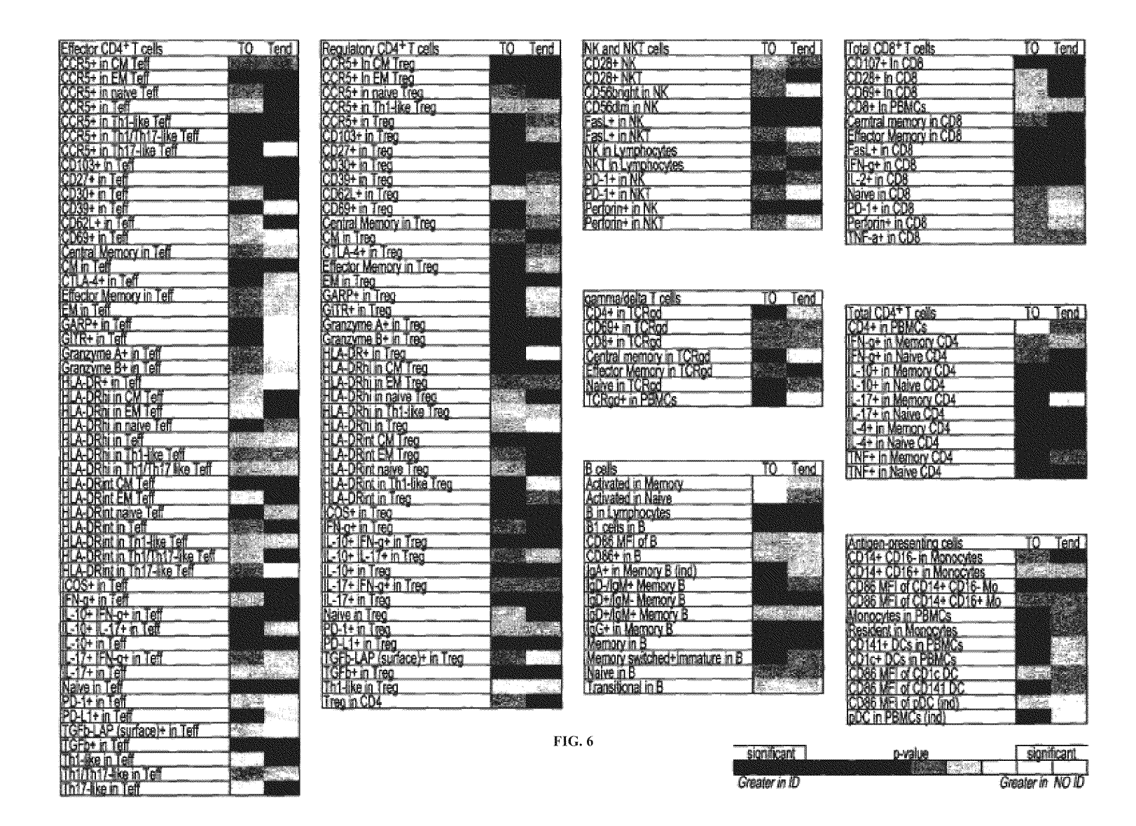

Ex vivo immunophenotyping by highly multiplexed using 11 concomitant parameters flow cytometry and functional assays, adding up to nearly 200 variables per sample. The samples were screened for JIA relevant factors, including adaptive and immune functions and subsets (FIG. 6).

Specifically, PBMCs from the clinical trial were thawed and a fraction immunophenotyped with a panel of markers using an 11-color LSR. For cytokine production, total PBMCs were stimulated with PMA/ionomycin for 6 hr, in the presence of GolgiPlug and GolgiStop for the last 4 hr, before performing the intracellular staining Sorted CD8+ T cells were stimulated for 6 hours with anti-CD3 crosslinked by P815 cells in the presence of CD107a antibody, GolgiPlug and GolgiStop. At the end of the experiment, the percentage of degranulating CD8+ T cells was assessed by the expression of CD107a. In addition, an intracellular staining for IFN-γ, TNF-α and IL-2 was performed on those cells. Other functional assays, not shown here, include proliferation and suppression assays of CD4+ T cells, activation of B cells via B cell receptor, CD40 and/or Toll-like receptors to determine proliferation and cytokine production, and NK cell degranulation in response to K562 targets. Data were stratified by time point and disease activity. (n=10 for ID, n=9 for no ID.)

Additionally, thawed PBMC samples from patients at T₀ and T_{end} were stained for CD4, CD25, FoxP3, GITR, CCR5, CD27, CCR6, CXCR3, CD161, HLA-DR, GARP and surface LAP without manipulation (n=10 for ID, n=9 for NO ID). Analyses were performed on gated CD4+CD25highFoxP3+ (T_{reg}) or CD4+CD25low/neg (T_{E}) cells. A portion of the cells were stimulated with PMA/ionomycin for 6 hr (in the presence of GolgiPlug and GolgiStop for the last 4 hr) before staining for intracellular IL-10 and IL-4. ID=PBMC from patients who reached ID at the end of the TREAT clinical trial; NO ID=PBMC from patients who did not reach ID at the end of the trial. *<=0.05, **<=0.01 ***<=0.001.

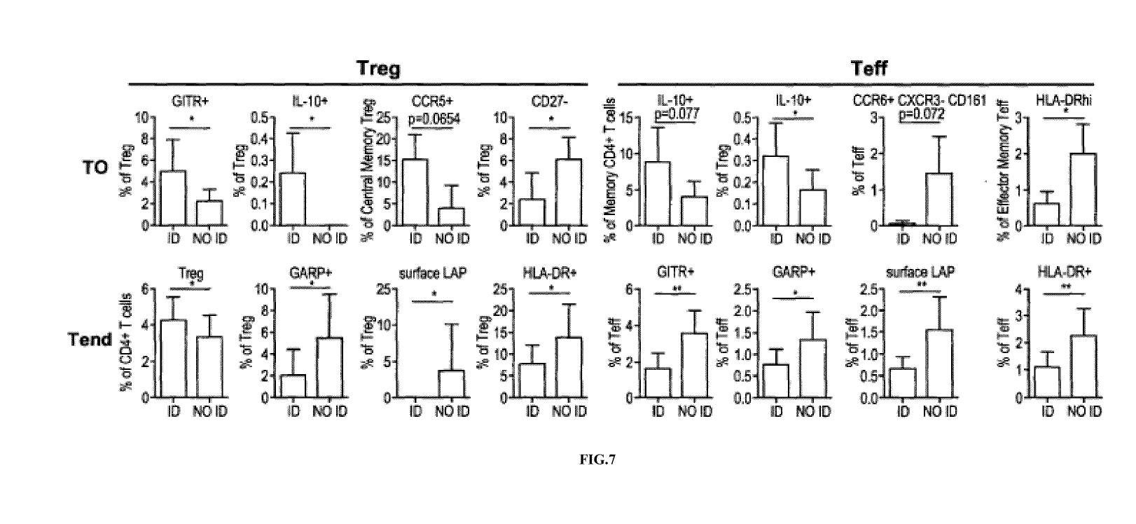

Several individual immune variables demonstrated differences based on time of sampling and clinical conditions. A majority of these variables grouped into meaningful signatures describing discrete subsets of T_{E} and T_{reg} (FIG. 7). These groups clearly indicate a predominance of a pro-inflammatory, activated phenotype of T_{E} in patients who did not respond to anti-TNFα antibody at both T₀ and T_{end}. In contrast, T_{E} from patients who achieved ID have a predominance of IL-10 and IL-4 at T₀. This data confirms that mechanisms of responsiveness to anti-TNFα antibody are strongly modulated by adaptive immunity

Additionally, T_{reg} is present at T₀ in patients who achieve ID. Conversely T_{reg} with an activated phenotype and positive for TGFβ are predominant at T_{end} in patients who do not achieve ID. This suggests that the T_{reg} is dynamic and that there may by a compensatory, but inefficient T_{reg} function, in patients who did not achieve ID.

### Example 5

The variables listed in Table 2 were selected for further analysis using clustering analysis, factorial analysis and multivariate logistical analysis to assess if multiple parameters in a group or a panel could improve predicting and identifying clinical outcomes.

TABLE 2

| Treg-T0 | Treg-Tend | Teff-T0 | Teff-Tend |
|---|---|---|---|
| Treg in CD4 | HLA-DR | CD69 | IL-10 |
| CCR5 | CD39 | CTLA-4 | IL-17 |
| CD27 | GARP | GARP | IL-4 |
| ICOS | GITR | GITR | TGF-b |
| IFN-g | Granzyme A | Gransyme A | TNF-a |
| IL-10 | PD-L1 | HLA-DR | |
| TGF-b | CD62L | IFN-g | |
| GITR | CD27 | IL-10/IL-17 | |
| GARP | IL-10 | IL-10 | |
| Surface LAP | IL-17 | IL-17 | |
| HLA-DR | IL-10/IFN-g | IL-4 | |
| PD-1 | TGFb | PD-1 | |
| | CCR | TGF-b | |
| | | Surface LAP | |

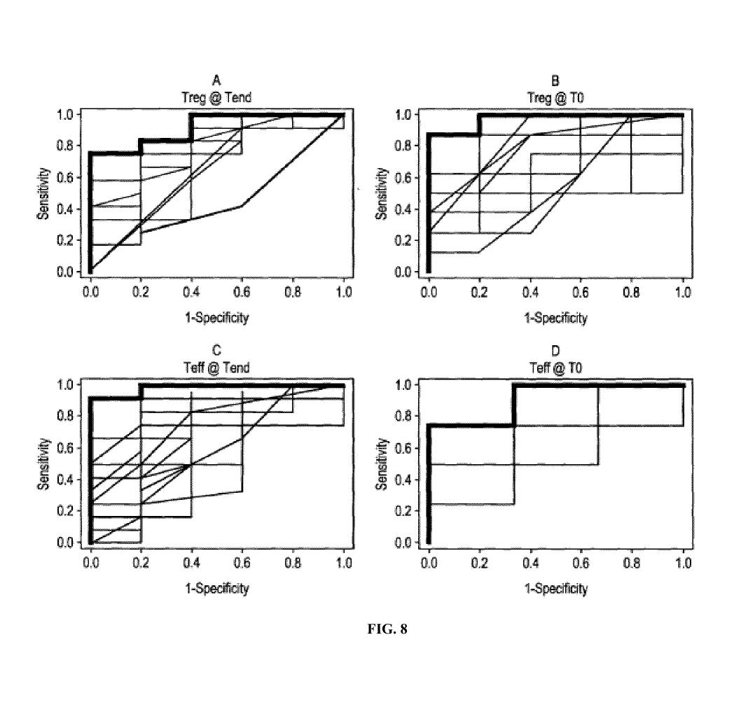

In FIG. 8 A-D receiver operating characteristic (ROC) curves are plotted using both univariate logistical regression models (one parameter at a time) and multivariate logistical regression models (a panel of multiple parameters at the same time). The plots show the ROC curves under multivariate analysis performed better than those under univariate analysis.

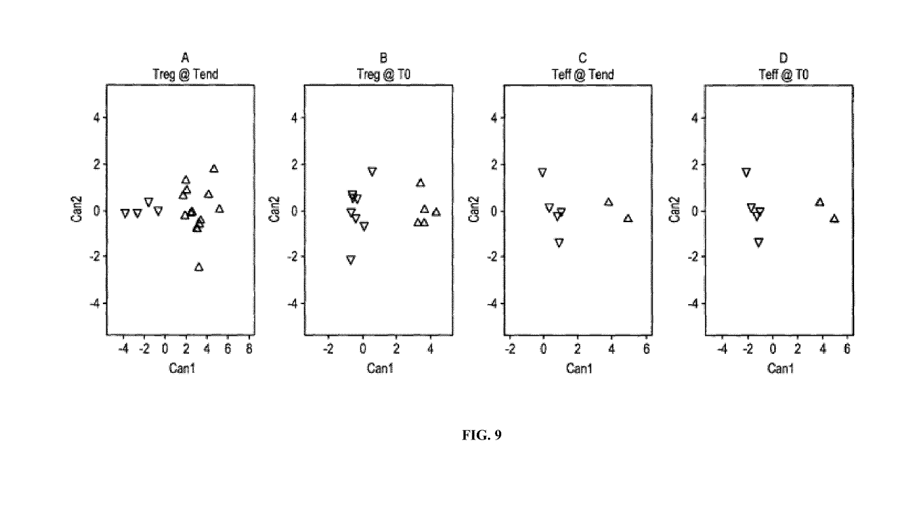

Scatter plots in FIG. 9 show patients can be separated into two clusters using each panel of multiple parameters at a time. The results indicate that patients can be divided into two clusters using each panel a of multiple parameters at a time.

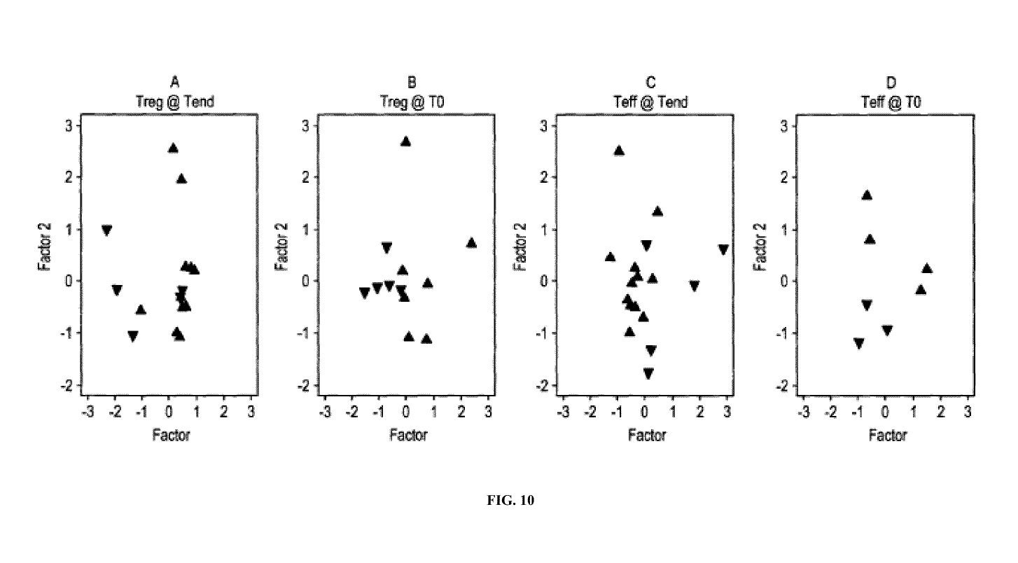

Scatter plots in FIG. 10 are from factorial analyses, suggesting ID and no ID patients can be reasonably separated by using two factors.

These data suggest that ID and no ID patients can be reasonably separated using two factors.

Accuracy of identifying ID (or no ID) was assessed using AUC, sensitivity and specificity respectively. Table 3 showed most of the panels reached over 85% AUC's using different approaches, suggesting that it is possible to improve prediction and discrimination by utilizing multiple sources of information through a panel of parameters rather than a single parameter.

TABLE 3

| Method | Panel | AUC | Sens | Spec |
|---|---|---|---|---|
| Clustering Analysis | A: Treg-Tend | 0.89 | 91.7% | 80.0% |
| | B: Treg-T0 | 0.90 | 87.5% | 80.0% |
| | C: Teff_Tend | 0.78 | 91.7% | 60.0% |
| | D: Teff-T0 | 0.83 | 100.0% | 67.0% |
| | A: Treg-Tend | 0.87 | 75.0% | 80.0% |
| Factor Analysis | B: Treg-T0 | 1.00 | 100.0% | 100.0% |
| | C: Teff_Tend | 0.95 | 100.0% | 80.0% |
| | D: Teff-T0 | 1.00 | 100.0% | 100.0% |
| Multivariate Logistical Model | A: Treg-Tend | 0.92 | 83.3% | 80.0% |
| | B: Treg-T0 | 0.98 | 100.0% | 80.0% |
| | C: Teff_Tend | 0.98 | 100.0% | 80.0% |
| | D: Teff-T0 | 0.92 | 75.0% | 100.0% |

Although the invention has been described with reference to the above example, it will be understood that modifications and variations are encompassed within the spirit and scope of the invention. Accordingly, the invention is limited only by the following claims.

### Example 6

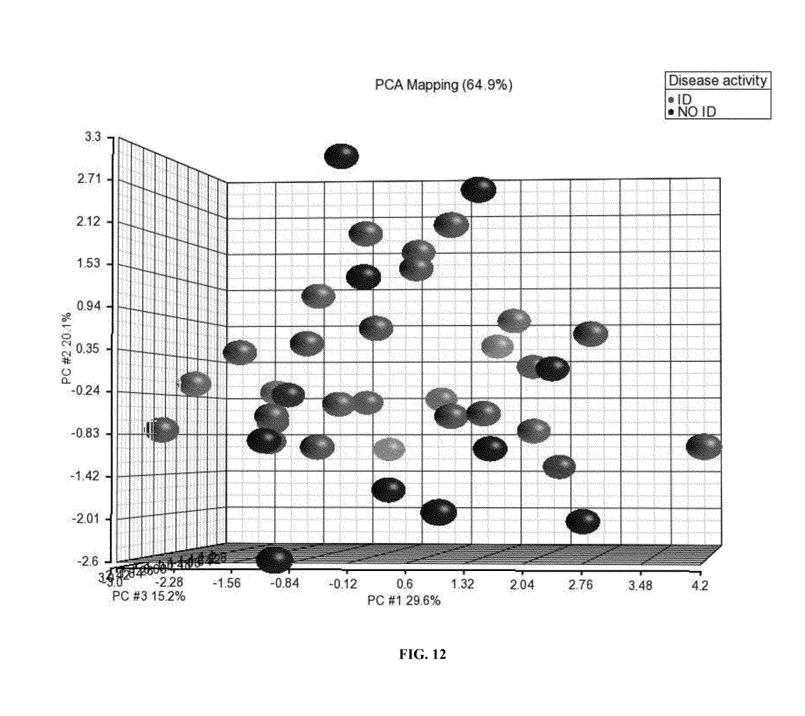

Patients were divided into “Responders” (ID) and “Non-responders” (NO ID) based on response to therapy (using Wallace's definition of clinical inactive disease (ID)). ID and NO ID patients had similar clinical presentations (described below) before starting treatment in the trial. The PCA graph (FIG. 12) visually demonstrates that clinical manifestations alone are unable to predict or confirm treatment response.

Principal Component Analysis (PCA) of Clinical Parameters.

Clinical parameters (ANA, anti-nuclear antibody titer; RF, rheumatoid factor titer; ESR, erithrocyte sedimentation rate; AJC, active joint count; PGA, physician global assessment; LOM, limitation of movement; HRQOL, health-related quality of life) measured at baseline for all available patients in group 1 were used for a principal component analysis (PCA), which reduces the complexity of a multi-parametric dataset to only three principal components (PCs). The length of each axis is proportional to the variance accounted for by the corresponding PC. Red and blue dots indicate ID and NO ID patients, respectively.

We identified candidate markers based on associations with known immunological functions. Our panel was comprehensive, including antigen-presenting cells, B cells, T cells, NK cells, NKT cells. This approach led to a extensive targeted dataset. This approach provided deep phenotypical and functional analysis of immune functions in the cohort studied.

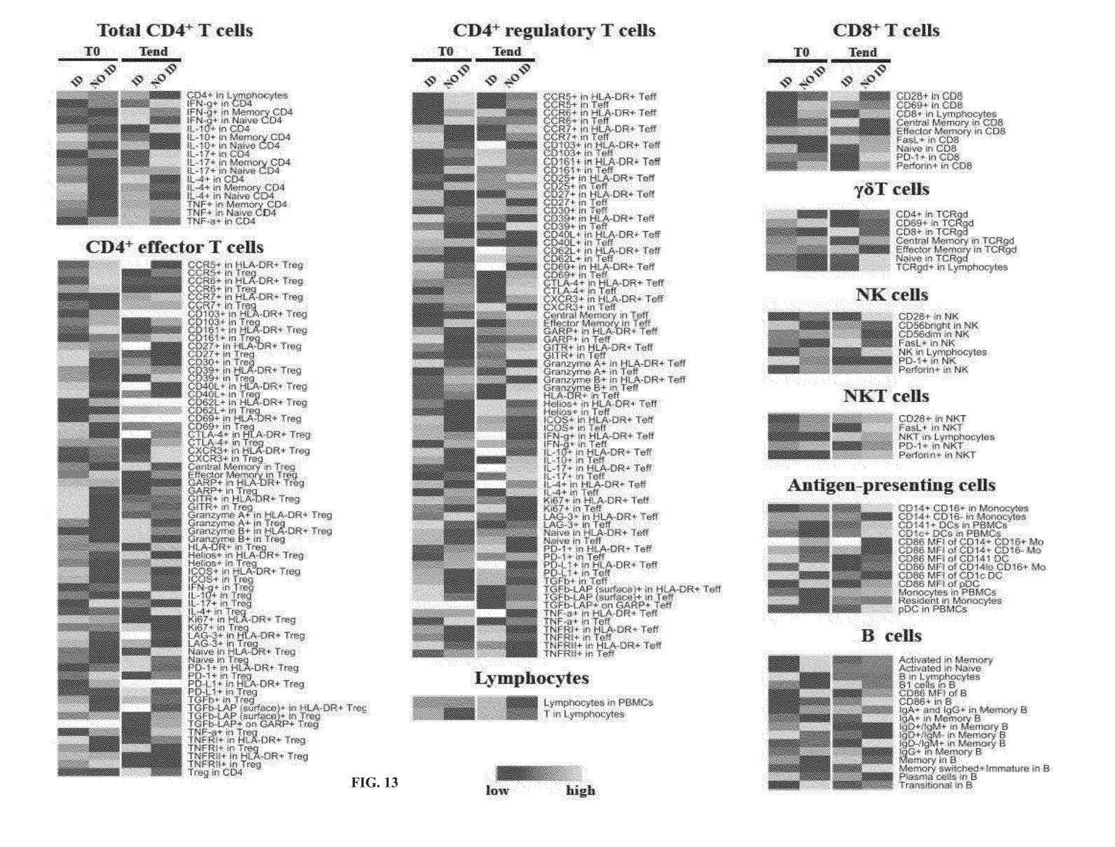

FIG. 13 Shows Complete Dataset of Predictive (T0) and Confirmatory Variables (Tend).

Primary measurements, stratified by time point (T0 and Tend) and disease activity (ID vs NO ID) were averaged, then normalized across rows (by parameter) before being color-coded according to the key. A total of 213 parameters were measured in up to 15 patients per group. White (blank) spots indicate no measurements were taken for that parameter at that specific time point.

Deep immuno-phenotyping returned 213 parameters that associated with treatment response at T0 and Tend (FIG. 13). An inherently pro-inflammatory phenotype (T cells) can be seen in NO ID patients at T0 and Tend, which could indicate resistance to therapy in non responders. The entire data set was analyzed for statistical significance (p<0.05) or near significance (p<0.1).

Predictive Variables

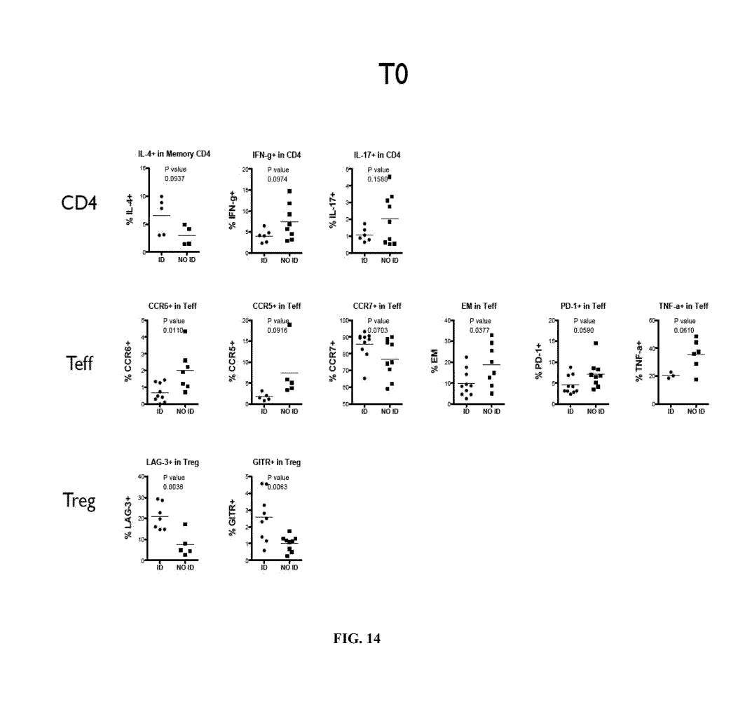

From T0, individual variables were further identified. Examples of statistically significant variables are shown below (FIG. 14).

Significant or Nearly Significant Parameters of CD4+ T Cells at Baseline (T0, Predictive).

Samples from the TREAT trial, collected before the therapy (T0), were analyzed. PBMCs were thawed and a fraction was immediately immunophenotyped by flow cytometry. Another portion of the PBMC was rested for 16 hours before being stimulated with PMA/ionomycin to induce cytokine production. All variables measured in CD4+ T cells at T0, achieving significance or near significance (p<0.1) and associated with a specific function are shown. Each dot corresponds to a single patient, and the line represents the mean. P-values have been determined with an unpaired two-tailed equal variance t-test.

| | | Sensitivity | Specificity |
|---|---|---|---|
| Parameter | AUC | (%) | (%) |
| CCR6+ in Teff | 0.84 | 67 | 86 |
| CCR7+ in HLA-DR+ Teff | 0.89 | 78 | 86 |
| CCR7+ in Teff | 0.83 | 78 | 86 |
| Naive in HLA-DR+ Teff | 0.93 | 67 | 86 |
| PD-1+ in HLA-DR+ Teff | 0.79 | 67 | 86 |
| PD-1+ in Teff | 0.78 | 56 | 86 |
| CD161+ in Teff | 0.86 | 89 | 86 |
| Effector Memory in Teff | 0.83 | 78 | 86 |
| GARP+ in Teff | 0.75 | 33 | 86 |
| GITR+ in Treg | 0.87 | 78 | 86 |
| CLUSTER (3 parameters in blue) | 0.97 | 89 | 100 |

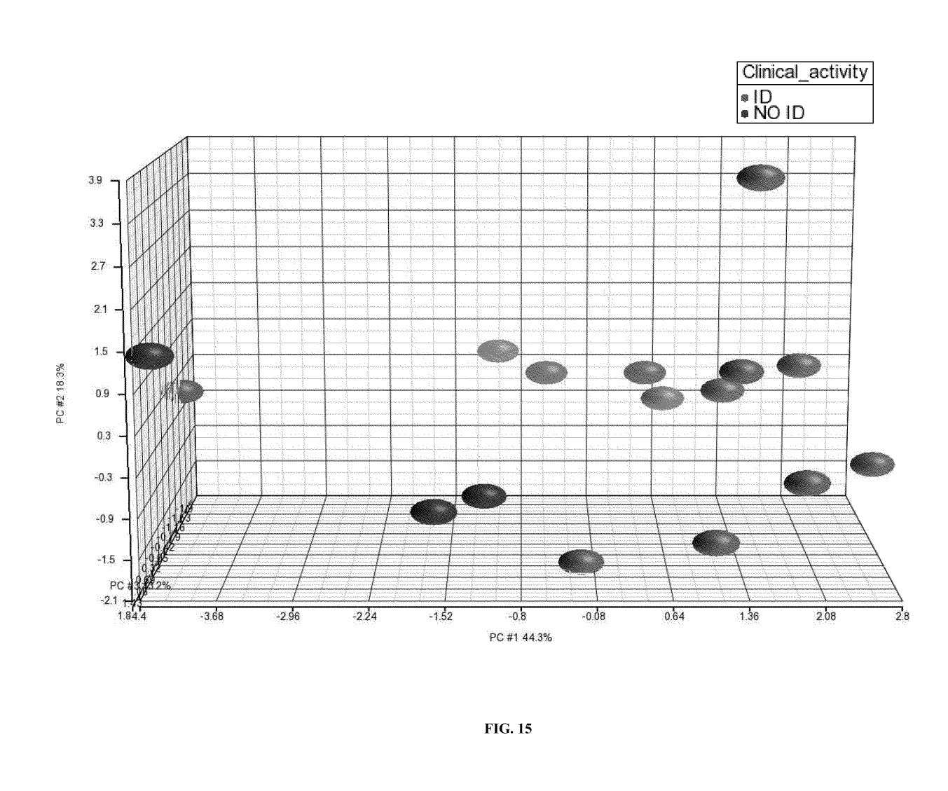

A visual representation is offered by Principal Component Analysis (PCA), showing how the populations of ID and NO ID patients are separated by our biomarker cluster is shown in FIG. 15.

Predictive Power of the Biomarkers (T0).

The parameters listed in Predictive Variables Table (derived from experiments described elsewhere) measured at T0 were included in a PCA. Red dots, ID; blue dots, NO ID.

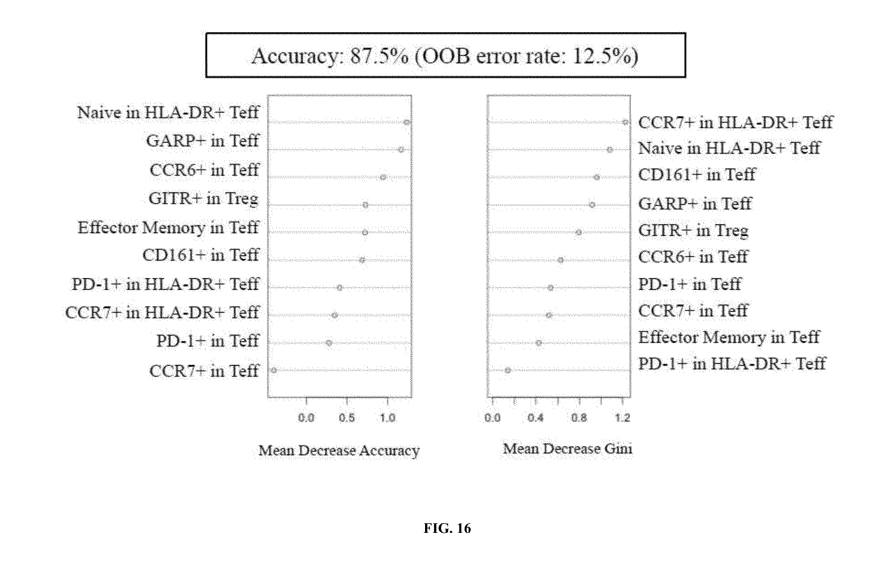

Clusters predicted ID and NO ID patients with an overall accuracy of 87.5% (FIG. 16).

Predictive Power of the Biomarkers (T0)

All the parameters listed in the Predictive Variables Table were fed to a random forest classifier. The figure depicts the Mean Decrease Accuracy and the Mean Decrease of Gini factor (a measure of variable importance) for each parameter of the cluster. OOB (out-of-bag) error rates were obtained as measure of random forest performance in classification accuracy.

Together, these data verify that when individual parameters are clustered, they inform an immune function. Example of immune functions determining clinical outcomes: when we stratified for resistance to therapy (NO ID), a subset of T cells with a clear pro-inflammatory signature emerges as a predictor. Increased production of pro-inflammatory Th1/Th17-type cytokines, an increased proportion of effector memory (EM) and exhausted PD-1+Teff, and a higher expression of CCR5 and CCR6, but lower expression of CCR7 (consistent with an inflammation-skewed profile of chemokine receptors) are present in patients at the beginning of treatment who will have NO ID at the end of their treatment course.

Confirmatory Variables

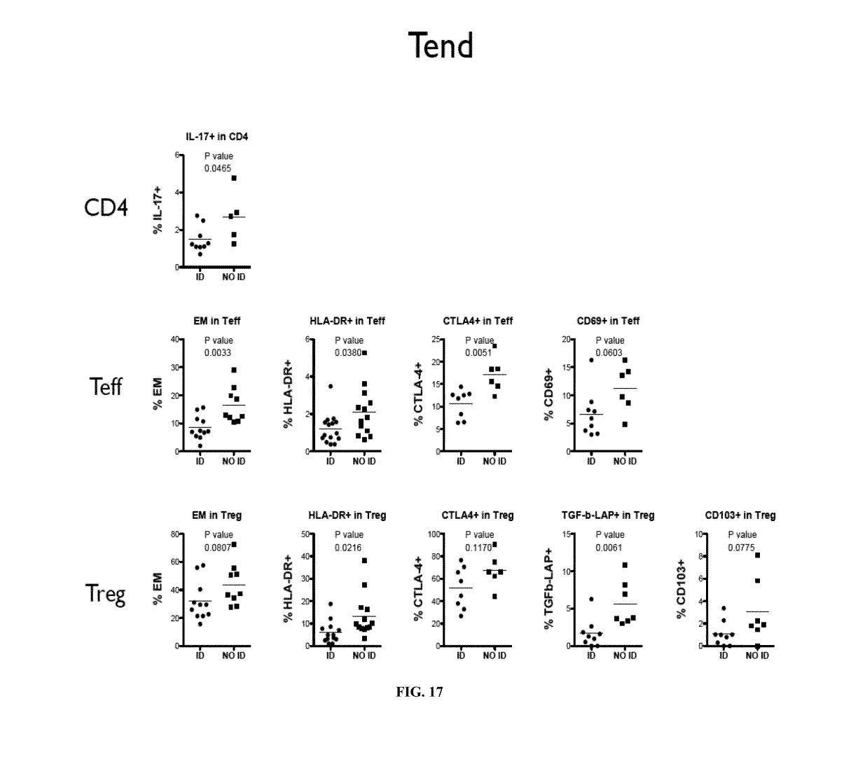

From Tend, individual variables were further identified. Examples of statistically significant variables are shown below (FIG. 17).

FIG. 17 Shows Significant or Nearly Significant Parameters of CD4+ T Cells at End of Study (Tend, Confirmatory).

Samples from the TREAT trial, collected at the end of study (Tend), were analyzed. PBMCs were thawed and treated as described above. All variables measured in CD4+ T cells at Tend, achieving significance or near significance (p<0.1) and associated with a specific function are shown. Each dot corresponds to a single patient, and the line represents the mean. P-values have been determined with an unpaired two-tailed equal variance t-test.

Sensitivity ranges from 22% to 89%, with an 86% specificity for all individual variables.

| | | Sensitivity | Specificity |
|---|---|---|---|
| Parameter | AUC | (%) | (%) |
| HLA-DR+ in Teff | 0.83 | 56 | 86 |
| HLA-DR+ in Treg | 0.88 | 89 | 86 |
| Naive in Teff | 0.70 | 56 | 86 |
| TGFb-LAP+ in Teff | 0.92 | 78 | 86 |
| TGFb-LAP+ in Treg | 0.94 | 89 | 86 |
| CD103+ in Treg | 0.75 | 78 | 86 |
| CD27+ in Teff | 0.85 | 78 | 86 |
| CD4+ in ICS Cells | 0.76 | 22 | 86 |
| CD69+ in Teff | 0.79 | 78 | 86 |
| CTLA-4+ in Teff | 0.87 | 78 | 86 |
| Effector Memory in Teff | 0.92 | 89 | 86 |
| Effector Memory in Treg | 0.84 | 56 | 86 |
| CLUSTER (3 parameters in blue) | 0.98 | 100 | 86 |

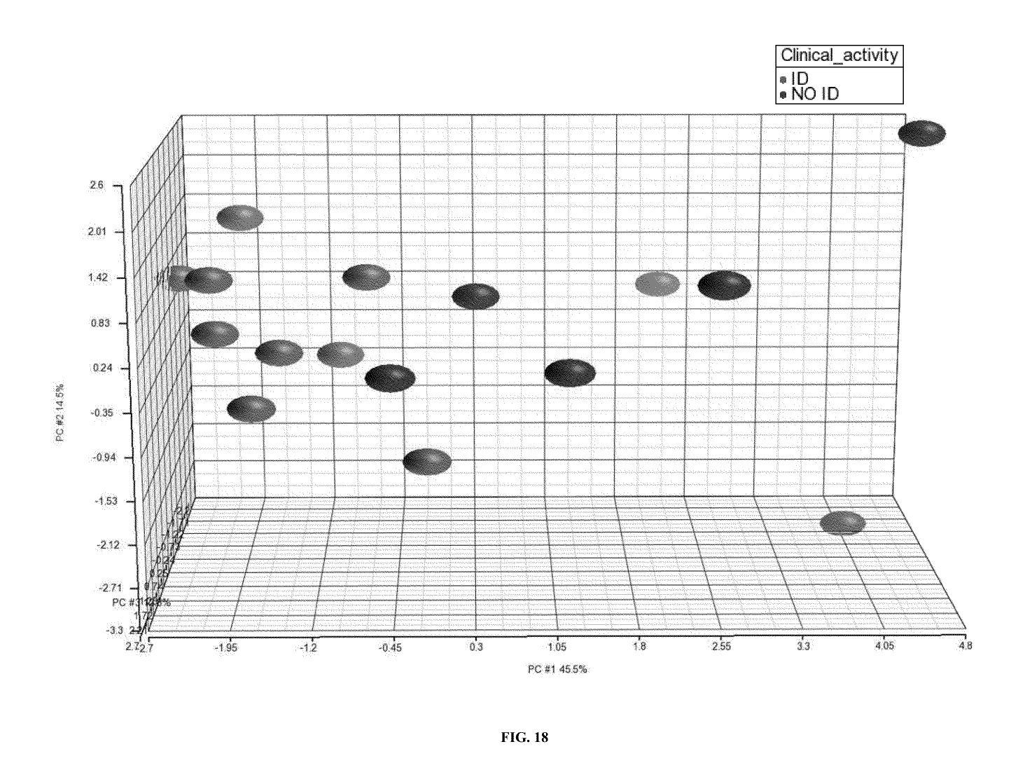

A visualization is offered by a PCA representation of how the populations of ID and NO ID patients separated by the clusters versus clinical parameters (FIG. 18).

Confirmatory Power of the Biomarkers (Tend).

The individual variables listed in panel B were included in a PCA. Red dots, ID; blue dots, NO ID.

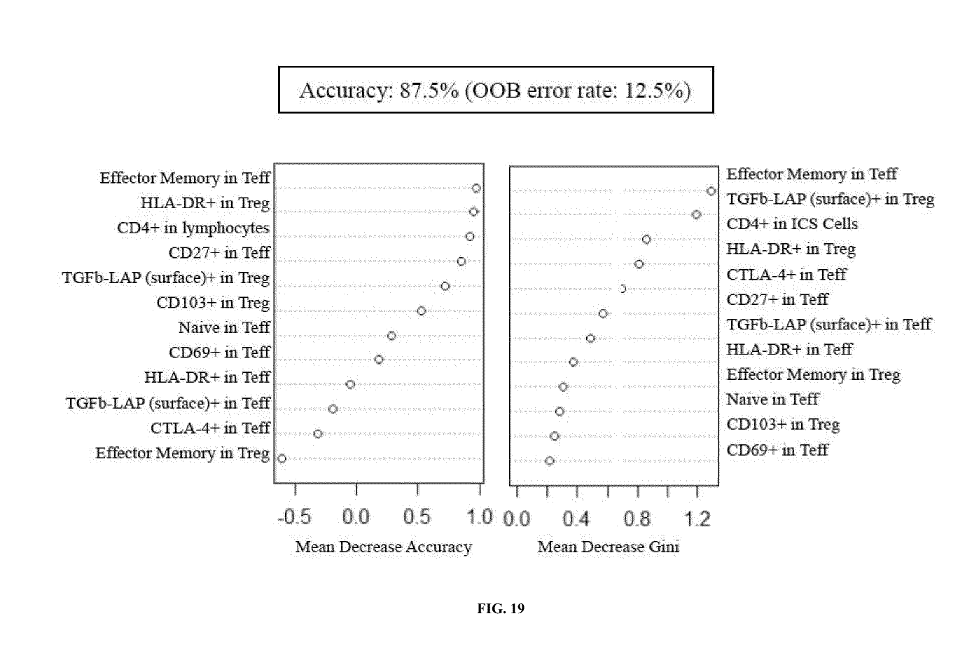

Once again, clustering parameters improved the accuracy of classification, segregating ID and NO ID patients with an overall accuracy of 87.5% (FIG. 19).

Confirmatory Power of the Biomarkers (Tend).

The parameters listed in the Confirmatory Variables Table were fed to a random forest classifier. The figure depicts the Mean Decrease Accuracy and the Mean Decrease of Gini factor (a measure of variable importance) for each parameter of the cluster. OOB (out-of-bag) error rates were obtained as measure of random forest performance in classification accuracy.

These data demonstrate the capabilities of the Immunomics platform to determine clusters of mechanistically cogent immune parameters. Example: an increased expression of well-known activation markers in both Treg (surface TGF-β/LAP, HLA-DR, CD103) and Teff (CTLA-4, CD69, HLA-DR) are present, in addition to an increased ratio of EM T cells over naive T cells in patients who have NO ID at the end of their treatment course. Altogether the data support the notion that hyper-activated T cells are resistant to treatments used in these NO ID patients.

Validation:

Results were analyzed and validated using a variety of statistical methods, including an internal validation method, based on a random forest classifier, which returns an “out-of-bag” (OOB) error rate, a parameter close to the n-fold cross-validation error rate. The OOB error rate at both T0 and Tend was 12.5% (FIGS. 4c-6c), revealing an excellent predictive and confirmatory value for our clusters in discriminating the disease activity in pJIA patients.

### Example 7

### Materials and Methods

Patients

The Trial of Aggressive Therapy (TREAT) included patients recruited in 17 US centers with new onset poly-articular JIA (≦12 mos), which were randomized either to receive aggressive therapy (prednisolone 0.5 mg/kg/d with taper, Methotrexate 0.5 mg/kg/week, Etanercept 0.8 mg/kg/week up to 50 mg/wk) or standard therapy (placebo tablets, Methotrexate 0.5 mg/kg/wk, placebo injection). Patients who achieved a PediACR70 at 4 months stayed in the arm to which they were assigned. Patients who did not achieve a PediACR70 at 4 months went into the combination arm using open label therapy regardless of which arm they had originally been in. Patients who remained in the double blind portion of the study until 6 months were assessed and if they had achieved inactive disease (ID), defined as in ref, they remained in the trial for an additional six months in a blinded fashion. If ID was achieved earlier than 6 months, they were followed for only 6 months from the time they achieved ID. Patients who did not achieve ID at 6 months went into open label combination therapy, similarly to those who had not achieved a PediACR70 at 4 months. Patients who flared after going into open label were removed from the trial. Each of the TREAT medical centers followed standard operating procedures (SOP) to collect whole blood by venipuncture and shipped samples overnight. For this study, blood drawn at baseline (T0) and after at least 4 months of Etanercept (Tend) has been used.

Isolation and Cryopreservation of PBMCs

EDTA-anticoagulated blood was received and processed within 24 hours from withdrawal. Blood was diluted in PBS and layered over half a volume of Histopaque-1077 (Sigma-Aldrich) at room temperature. PBMCs were collected at the interface between upper and lower layers after centrifuging at 700×g for 24 minutes with the brake off. PBMCs were washed three times to eliminate any residual Histopaque and contaminant platelets. Then, they were frozen at −80° C. in freezing medium (90% Fetal Bovine Serum, 10% DMSO) in a Mr. Frosty container (Nalgene) at 10-20×106 cells/ml. The following day, cryovials were transferred to liquid nitrogen for long-term storage. Viability by Trypan Blue exclusion was routinely above 90% before freezing, and greater than 85% after thawing.

Cell Culture

Cells were cultured in complete medium: RPMI-1640 (HyClone) supplemented with 5% heat-inactivated Human Serum AB (GemCell), 2 mM L-Glutamine (Gibco) and 100 U penicillin/100 μg streptomycin (Gibco).

Proliferation and Suppression Assays

PBMCs were thawed and allowed to rest for 16 hours in complete medium supplemented with 20 IU/ml recombinant IL-2. Cells were then stained with antibodies for CD4, CD25 and CD127 and sorted as regulatory T cells (Treg) (CD4+CD25highCD127−) or effector T cells (Teff) (CD4+CD25−/low) with a FACSAria II (BD Biosciences). Sorted Teff were labeled with CFDA-SE (Life Technologies) according to the manufacturer's instructions. 5,000 Teff were stimulated with 500 anti-CD3/CD28 beads (Life Technologies) for 5 days, in the presence or absence of 5,000 Treg. At day 5, cells were harvested and the dilution profiles of CFDA-SE were analyzed by flow cytometry. The proliferation index (PI) was calculated using ModFit LT (Verity Software House).

Immunophenotyping and Cytokine Secretion

Ex vivo immunophenotyping was performed on PBMCs right after thawing. Dead cells were excluded using Live/Dead Fixable Near-IR Stain (Life Technologies). Fc receptors (FcR) were blocked using FcR blocking reagent (Miltenyi Biotec) to avoid non-specific antibody binding. Intracellular staining was performed with the FoxP3 buffer set from eBioscience, following manufacturer's instructions. Samples were acquired with an LSRFortessa (BD Biosciences). To measure cytokine production, total PBMCs were thawed and allowed to rest for 16 hours in complete medium before stimulation with 20 nM PMA and 200 nM ionomycin (PMA/iono) for 6 hours. 2 hours after PMA/iono stimulation, GolgiPlug and GolgiStop (BD Biosciences) were added to cell culture, following manufacturer's instructions. CD3+CD4+CD25+FoxP3+ Treg and CD3+CD4+CD25−/lowFoxP3− Teff were gated for subsequent analysis. Fluorochrome-conjugated antibodies were from Biolegend, BD Biosciences and eBioscience. Analysis was performed with FlowJo (Treestar). The stability of FoxP3 expression upon PMA/iono stimulation is shown in FIG. S3.

To normalize the measurements of markers tested in multiple batches, three normal donors were stained with the panels of markers used in each batch. A linear regression analysis was run (R2 consistently higher than 0.9) and the slope was used as a normalizing coefficient.

Statistical Modeling

The dataset was first cleaned by preserving only variables measured in at least 3 ID and 3 NO ID patients. As a result, 169 measurements on up to 21 patients (T0) or 147 measurements on up to 28 patients (Tend) were obtained. Significant or nearly-significant measurements distinguishing clinical activity between ID and NO ID were enriched using t-tests (with equal or unequal variance) and the Mann-Whitney U-test by requiring p<0.1 in any of the three. Surviving variables were grouped in a cluster according to data availability, so to minimize imputation of missing data. When necessary, a conservative and unbiased imputation using the variable mean from the complete dataset (with no distinction between ID and NO ID) was performed. As a result, two datasets (one for each time point) were generated. Cluster variables, either taken singularly or all together, were used in univariate and multiple logistic regression models, respectively. For each model, we built receiver operating characteristic (ROC) curves and we determined the area under curve (AUC). To avoid overfitting, multiple logistic regression was also built using only the three best performing individual variables in terms of AUC. The optimal threshold of the log odds ratio was determined by the highest sensitivity under the imposition of a specificity >80%. Principal Component Analysis was performed on the reduced datasets of cluster variables or on clinical data (anti-nuclear antibody, rheumatoid factor, erythrocyte sedimentation rate, active joint count, physician global assessment, limitation of movement and health-related quality of life). The reduced datasets were also fed to a Random Forest classifier, which uses an internal validation method by training the classification and regression trees through bootstrapping on 63.2% randomly selected data and validating predictions using the rest of the 36.8% “out of bag” (OOB) data. The OOB error rate was used as an indicator of the performance of the model.

In column graphs and scatter plots, the error bars represent the standard deviation (SD), and p-values were calculated with an unpaired two-tailed equal variance t-test.

Statistical analysis was performed with SAS (SAS Institute), R (CRAN) with Bioconductor, Partek Genomics Suite (Partek) and Weka (University of Waikato).

1-9. (canceled)

10. A method for predicting a response to an immune mediated disease therapeutic agent comprising:
a) isolating proliferating blood mononuclear cells (PBMCs) from a subject treated for an immune mediated disease;
b) stimulating the PBMCs;
c) identifying subpopulations of PBMCs;
d) comparing data from step (c) to subject response to the therapeutic; and
e) selecting a marker profile related to a positive response to the an immune mediated disease therapeutic agent.

11. The method of claim 10, wherein the therapeutic agent is a biologic agent.

12. The method of claim 11, wherein the biologic agent is an antibody.

13. The method of claim 12, wherein the antibody is selected from the group consisting of: adalimumab, etanercept, infliximab, certolizumab, golimumab, anakinra, rituximab, abatacept, tocilizumab, muronomab, abciximab, daclizumab, basilimab, omalizumab, efalizumab, natalizumab, certolizumab pegol, usterkinumab, belimumab, clenoliximab, keliximab, priliximab, teneliximab, vapaliximab, ibalizumab, aselizumab, apolizumab, benralizumab, cedelizumab, certolizumab pegol, eculizumab, epratuzumab, erlizumab, fontolizumab, mepolizumab, ocrelizumab, pascolizumab, pexelizumab, reslizumab, rontalizumab, rovelizumab, ruplizumab, siplizumab, talizumab, teplizumab, tocilizumab, toralizumab, vedolizumab and visilizumab and any combination thereof.

14. The method of claim 10, wherein the immune mediated disease is selected from the group consisting of arthritis, atherosclerosis, diabetes, traumatic brain injury and depression.

15. The method of claim 10, wherein the stimulating agent is selected from the group consisting of: an anti CD3/CD28 antibody, Tetanus toxoid peptides, PMA, ionomycin or other appropriately selected antigens, known to be contributors to the inflammatory process or any combination thereof.

16. The method of claim 10, wherein the PBMC subpopulation identification comprises FACS analysis.

17. The method of claim 15 wherein, markers identified by FACS analysis are selected from the group consisting of: annexin V, B7-H1, B7-H3, B7-H4, B7-DC, B cells, memory B cells, CCR5, CCR6, CD1c, CD4, CD8, CD 11c, CD14, CD16, CD19, CD25, CD 27, CD28, CD30, CD39, CD40, CD56, CD62L, CD69, CD80, CD86, CD103, CD107, CD123, CD127, CD141, CD161, CD200, CTLA-4, CXCR3, Fas, FasL, FoxP3, GARP, GATA-3, GITR, GranzymeA, GranzymeB, HLA-DR, ICOS, IL-1β, IL-2, IL-4, IL-6, IL-10, IL-12, IL-12R, IL17, IL-17A, IL-21, IL-23, IFNγ, Ki67, KOS, memory CD4 cells, monocyte cells, naïve CD4 cells, NK cells, NKT cells, OX40, PD-1, PD-LI/II, Perforin, RORC, STAT-3 phosphorylation, STAT-5 phosphorylation, surface LAP, T-bet, TCR_{gd}, TGFβ, TNFα, T regulatory cells, T effector cells, Th1, Th17 and any combination thereof.

18. The method of claim 10, wherein the PBMC subpopulation identification is done by functional assay.

19. The method of claim 18, wherein the functional assay is selected from the group consisting of: a proliferation assay, a suppression assay, confocal microscopy, Western blot, CTV staining, CFSE staining and PI assay.

20. The method of claim 19, wherein confocal microscopy determines the phosphorylation state of STAT-3 and STAT-5.

21. The method of claim 10, wherein the PBMC subpopulation identification is by PCR analysis.

22. The method of claim 10, wherein the profile includes PCR analysis of markers selected from the group consisting of: FoxP3, GATA-3, Granzyme, ICOS, IκBα, IKK, IL-2, IL-4, IL-6, IL-10, IL-12, IL-12R, IL-17A, IL-21, IL-23, IFNγ, NFκb pathway, p50, PD-1, Perforin, RelA, RORC, T-bet, TGFβ, TNFα and any combination thereof.

23. The method of claim 10, wherein the subpopulation is selected from the group consisting of:
a) CD4⁺, CD127⁺ and T effector cells;
b) CD4⁺, CD127⁻ and T regulatory cells;
c) CD4⁺, CD 127⁻, CD25^{low/neg} and nT regulatory cells;
d) CD4⁺, CD127⁻, CD25⁺⁺ and nT regulatory cells;
e) CD4⁺, CD127⁻, CD25⁺⁺, PD-1⁺ and tT regulatory cells;
f) CD14, CD19, CD11c and CD123;
g) CD56⁺, CD3⁻ and NK cells;
h) CD8⁺ and T cells;
i) CD4⁺, CXC3⁺, CCR6⁻, and Th1 cells;
j) CD4⁺, CXC3, CCR6⁺, CD161⁺ and Th17 cells;
k) CD4⁺, CXC3⁺, CCR6⁺, CD161⁺, Th1 cells and Th17 cells;
l) CD8⁻, CD4⁺ and T cells;
m) CD8⁺, CD4⁻ and T cells;
n) CD 19⁺ and B cells;
o) CD14⁺ and monocytes;
p) CD4⁺, CD25^{high} and T effector cells;
q) CD4⁺, CD25⁻ and T effector cells; and
r) CD4⁻, CD8⁻, CD 19⁺ and B cells.

24. A method of monitoring the course of an immune mediated disease therapeutic agent therapy comprising:
a) isolating PBMCs from a subject previously or currently being treated for an immune mediated disease;
b) stimulating the PBMCs;
c) identifying subpopulations of PBMCs;
d) comparing data for step (c) to a subject response to the therapeutic agent; and
e) selecting a marker profile related to a positive response to the therapeutic agent, thereby monitoring the course of therapy in a subject.

25. Use of a Immunomics platform to predict response to an immune mediated disease therapeutic agent comprising:
a) isolating PBMCs from a subject treated for an immune mediated disease;
b) stimulating the PBMCs;
c) identifying subpopulations of PBMCs;
d) comparing data for step (c) to a subject response to a therapeutic agent; and
e) selecting a marker profile related to a positive response to therapeutic agent.

26. The method of claims 24 and 25, wherein the PBMCs are isolated from subjects treated with a biologic therapeutic.

27. A method of predicting response to an immune mediated disease therapeutic agent in a subject comprising:
a) obtaining a blood sample from a subject in need of therapy; and
b) analyzing the blood sample for a marker profile identified by the method of claim 10; wherein when markers associated with a positive response to the therapeutic agent are identified, a subject responsive to therapy is identified.

28. The method of claim 27, wherein the blood sample is analyzed using a method selected from the group consisting of:
a) a protein chip;
b) a gene expression chip;
c) protein expression;
d) detection of phosphorylation;
e) phosphorescence or luminesence;
f) immunobeads; or
g) a combination of all the above.

29. A protein or gene profile containing the markers identified in e) of claim 10.

30. An array containing one or more protein or gene markers of claim 29.

31. The array of claim 30 wherein the array is on a gene or protein chip.

32. A method for diagnosing an immune mediated disease comprising:
a) isolating proliferating blood mononuclear cells (PBMCs) from a subject diagnosed with an immune mediated disease;
b) stimulating the PBMCs;
c) identifying subpopulations of PBMCs;
d) comparing data from step (c) to subject diagnosis; and
e) selecting a marker profile related to a positive diagnosis of an immune mediated disease.

33. A method for predicting a positive response to a therapeutic agent comprising:
a) obtaining a blood sample from a subject in need of therapy; and
b) analyzing the blood sample for a signature marker profile; wherein when markers associated with a positive response to a TNFα therapeutic agent are identified, a subject responsive to the therapy is identified.

34. A method for predicting a negative response to a therapeutic agent comprising:
a) obtaining a blood sample from a subject in need of therapy; and
b) analyzing the blood sample for a signature marker profile; wherein when markers associated with a negative response to a TNFα therapeutic agent are identified, a subject non-responsive to the therapy is identified.

35. The method of claim 33 or 34, wherein the subject is diagnosed with human arthritis.

36. A signature marker profile for predicting response to a TNFα therapeutic agent wherein the profile contains clusters of immune variables selected from the group consisting of:
a) immune variables associated with T_{E} at T₀;
b) immune variables associated with T_{E} at T_{end};
c) immune variables associated with T_{reg} at T₀; and
d) immune variables associated with T_{reg} at T_{end} or any combination thereof.

37. The signature marker profile of claim 36, wherein the immune variables associated with T_{B} at T₀ comprises at least one of the markers selected from the group consisting of:
a) IL-4⁺ in Memory CD4;
b) IL-10⁺ in Memory CD4;
c) IL-4⁺ in Naive CD4;
d) IL-10⁺ in Naive CD4;
e) IL-17⁺ in Naive CD4;
f) TNFα⁺ in Naive CD4;
g) IL-10⁺ in T_{E};
h) TGFβ⁺ in T_{E};
i) Th1-like in T_{E};
j) Th17-like in T_{E};
k) IL-17⁺ in T_{E}; and
l) IFN-γ⁺ in T_{E}.

38. The signature marker profile of claim 36, wherein the immune variables associated with T_{E} at T_{end} comprises at least one of the markers selected from the group consisting of:
a) IL-4⁺ in Memory CD4;
b) IL-10⁺ in Memory CD4;
c) IL-4⁺ in Naive CD4;
d) IL-10⁺ in Naive CD4;
e) IFN-γ⁺ in Naive CD4;
f) TGFβ⁺ in T_{E};
g) GITR⁺ in T_{E};
h) GARP⁺ in T_{E};
i) TGFβ-LAP (surface)⁺ in T_{E};
j) HLA-DR⁺ in T_{E};
k) CD69⁺ in T_{E};
l) PD-1⁺ in T_{E};
m) CTLA-4⁺ in T_{E};
n) Granzyme A⁺ in T_{E}
o) Granzyme B⁺ in T_{E};
p) IL-17⁺ in T_{E};
q) IL-10⁺ in T_{E};
r) IL-10⁺ IL-17⁺ in T_{E}; and
s) IL-17⁺ in Memory CD4.

39. The signature marker profile of claim 36, wherein the immune variables associated with T_{reg} at T₀ comprises at least one of the markers selected from the group consisting of:
a) GITR⁺ in T_{reg};
b) GARP⁺ in T_{reg};
c) CD27⁺ in T_{reg};
d) CD39⁺ in T_{reg};
e) CD39⁺ in Memory T_{reg};
CCR5⁺ in CM T_{reg};
g) PD-L1⁺ in T_{reg};
h) Granzyme A⁺ in T_{reg};
i) HLA-DR in T_{reg};
j) HLA-DR^{int} in Th1-like T_{reg};
k) IL-10⁺ in T_{reg};
l) IL-10⁺ IFN-γ⁺ in T_{reg};
m) TGFβ⁺ in T_{reg};
n) CD62L⁺ in T_{reg};
o) Ki67⁺ in Memory T_{reg};
p) Th1-like in T_{reg}; and
q) IL-17⁺ in T_{reg}.

40. The signature marker profile of claim 36, wherein the immune variables associated with T_{reg} at T_{end} comprises at least one of the markers selected from the group consisting of:
a) T_{reg} in CD4;
b) CCR5⁺ in CM/EM T_{reg};
c) CD27⁺ in T_{reg};
d) ICOS⁺ in T_{reg};
e) IFN-γ⁺ in T_{reg};
l) IL-10⁺ in T_{reg};
g) TGFβ⁺ in T_{reg};
h) GITR⁺ in T_{reg};
i) GARP⁺ in T_{reg};
k) TGFβ-LAP (surface)⁺ in T_{reg};
l) HLA-DR^{+/hi} in T_{reg};
m) PD-1⁺ in T_{reg};
n) CD62L⁺ in T_{reg};
o) Th1-like in T_{reg}; and
p) IL-10⁺ IL-17⁺ in T_{reg}.

41. A signature marker profile for predicting response to a TNFα therapeutic agent wherein the markers are selected from the group consisting of: IL-4⁺ in Memory CD4, IL-10⁺ in Memory CD4, IL-4⁺ in Naive CD4, IL-10⁺ in Naive CD4, IL-17⁺ in Naive CD4, TNFα⁺ in Naive CD4, IL-10⁺ in T_{E}, TGFβ⁺ in T_{E}, Th1-like in T_{E}, Th17-like in T_{E}, IL-17⁺ in T_{E}, IFN-γ⁺ in T_{E}, IFN-γ⁺ in Naive CD4, GITR⁺ in T_{E}, GARP⁺ in T_{E}, TGFβ-LAP (surface)⁺ in T_{E}, HLA-DR⁺ in T_{E}, CD69⁺ in T_{E}, PD-1⁺ in T_{E}, CTLA-4⁺ in T_{E}, Granzyme A⁺ in T_{E}, Granzyme B⁺ in T_{E}, IL-10⁺ IL-17⁺ in T_{E}, IL-17⁺ in Memory CD4, GITR⁺ in T_{reg}, GARP⁺ in T_{reg}, CD27⁺ in T_{reg}, CD39⁺ in T_{reg}, CD39⁺ in Memory T_{reg}, CCR5⁺ in CM T_{reg}, PD-L1⁺ in T_{reg}, Granzyme A⁺ in T_{reg}, HLA-DR in T_{reg}, HLA-DR^{int} in Th1-like T_{reg}, IL-10⁺ in T_{reg}, IL-10⁺ IFN-γ+ in T_{reg}, TGFβ⁺ in T_{reg}, CD62L⁺ in T_{reg}, Ki67⁺ in Memory T_{reg}, Th1-like in T_{reg}, IL-17⁺ in T_{reg}, T_{reg} in CD4, CCR5⁺ in CM/EM T_{reg}, ICOS⁺ in T_{reg}, IFN-γ⁺ in T_{reg}, IL-10⁺ in T_{reg}, TGFβ-LAP (surface)⁺ in T_{reg}, HLA-DR^{+/hi} in T_{reg} and IL-10⁺ IL-17⁺ in T_{reg}.

42. The signature of claim 40, wherein the signature predicts the response of a subject diagnosed with human arthritis to the TNFα therapeutic agent.

43. A method of predicting response to a therapeutic agent comprising:
a) obtaining a blood sample from a subject in need of therapy; and
b) analyzing the blood sample for a the methylation pattern of the genes listed in Table 5; wherein when a methylation pattern associated with a positive response to a TNFα therapeutic agent is identified, a subject responsive to the therapy is identified.

44. A method of predicting response to a therapeutic agent comprising:
a) obtaining a blood sample from a subject in need of therapy; and
b) analyzing the blood sample for a the methylation pattern of the genes listed in Table 5; wherein when a methylation pattern associated with a negative response to a TNFα therapeutic agent is identified, a subject non-responsive to the therapy is identified.
